(19) United States
(12) Patent Application Publication
(10) Pub. No.: US 2025/0057642 A1
(43) Pub. Date: Feb. 20, 2025

(54) Immunoprotective Type Implantable Cell Delivery Device

(71) Applicants: Universiteit Maastricht, Maastricht (NL); Academisch Ziekenhuis Maastricht, Maastricht (NL)

(72) Inventors: Aart Alexander Van Apeldoorn, Maastricht (NL); Denise Francoise Antoinette De Bont, Maastricht (NL)

(21) Appl. No.: 18/722,298

(22) PCT Filed: Dec. 21, 2022
(86) PCT No.: PCT/EP2022/087335

§ 371 Date: Jun. 20, 2024

(30) Foreign Application Priority Data
Dec. 23, 2021 (EP) 21217479.1

(51) Int. Cl. A61F 2/02

(52) U.S. Cl. CPC A61F 2/022; A61F 2220/0058

(57) ABSTRACT

The present invention relates to an immunoprotective type implantable cell delivery device for implanting cells in a subject comprising an enclosed space between a top and bottom porous membrane and a mesh. The mesh is used to prevent clustering of the cells. The invention further relates to a method of constructing the closed type implantable cell delivery device. The invention also relates to the device for use in a method of treatment.

### FIELD OF THE INVENTION

The invention relates to the field of implantable cell delivery devices, particularly implantable cell delivery devices of the immunoprotective type, which allow isolation of the implanted cells from the host immune cells. The invention particularly relates to immunoprotective devices comprising a mesh for embedding cells. Such devices may be used for implanting cells such as pancreatic islet cells in a subject. The invention further relates to a method for constructing the device and a method of loading the device with cells. Further, the invention is related to the use of the closed type implantable cell delivery device containing cells in the treatment of a disease or disorder by implanting the device in a subject.

### BACKGROUND

Transplantation of donor cells in patient holds a promising tool for the treatment of a variety of diseases. For example, pancreatic islet cells may be transplanted in diabetes patients.

Type 1 diabetes (T1D) is an autoimmune disease where the insulin producing β-cells, in the islets of Langerhans, are destroyed by the immune system. It is estimated that 20 to 40 million people worldwide have T1D. Insulin administration need to be tightly controlled to prevent hypo or hyperglycaemia as these events can result in tissue and organ damage which can be life-threatening if left untreated. Most T1D patients are prescribed with daily exogenous insulin, however tight control of blood glucose levels remains difficult to achieve due to insufficient dose titration and poor patient compliance. One method to obtain normal glycaemic blood glucose levels without insulin is by clinical islet transplantation (CIT). CIT is a minimally invasive therapy with few complications and with the benefit that patients can become insulin independent for approximately five years. The islets are isolated from a donor pancreas and transplanted into the recipient's hepatic portal vein. However, due to donor shortage and the need of long-life immunosuppressive drugs to prevent any alloimmunity or autoimmune reoccurrence, CIT is only available for patients with poorly controlled diabetes. The efficiency of CIT is limited, as 50-70% of the transplanted islets are lost shortly after transplantation due to an instant blood-mediated inflammatory reaction (IBMIR). IBMIR is triggered by the exposure of islets directly to blood and results in rapid activation and binding of platelets to islets. Islets will then be encapsulated in blood clots, finally leading to a loss of transplanted cells. The use of an extrahepatic islet delivery device such as a closed (immunoprotective) device, can improve cell delivery, as it avoids IBMIR and the recipient does not need immunosuppressive drugs since the cells inside the device are not accessible to the immune system.

A drawback of existing closed (immunoprotective) type devices is that the cells or cell clusters tend to aggregate inside the device. For example, US 2021/022846 describes a closed device for implanting cells, however no means are described to prevent cells from clumping together. Aggregation of cells tends to cause necrosis of the cells at the center of the cell mass due to deprivation of nutrients and oxygen. A way to prevent aggregation is embedding the cells (or cell clusters) in a hydrogel inside the immunoprotective device. However, the drawback of embedding in hydrogel is that this, although preventing clustering and resulting necrosis, still hinders diffusion of nutrients, gases and proteins. This renders the diffusion capacity of such immunoprotective type devices too low, resulting in devices that cannot contain enough cells for the intended therapy (e.g. islet-like cell transplantation in type 1 diabetes). Further, hydrogels hamper the diffusion of secreted factors (e.g. insulin in the case of islet cells) out of the implant device. For these reasons, among other, improved immunoprotective type devices are needed.

Additionally, WO 2018/114098 discloses an open type device in which a mesh may be used to prevent cells from moving or aggregating. U.S. Pat. No. 6,008,049 discloses a cell culture device in which a mesh is used. US 2020/360566 describes a closed type device for implantation in low oxygen sites which uses beads to which cells adhere in order to prevent clumping. These documents describe methods to prevent cells from aggregating, e.g., by using meshes, however none of these documents describe suitable ways to implement these in a closed device such as to allow diffusion of oxygen and nutrients, and still enabling even seeding of the cells in the device.

The present invention addresses the above problems, among others, by the immunoprotective type device and methods as defined in the appended claim.

### SUMMARY OF THE INVENTION

In a first aspect, the invention relates to an implantable device of the immunoprotective type for implanting cells or cell aggregates in a subject, wherein the device comprises a bottom membrane and a top membrane, wherein the bottom membrane and the top membrane are joined together with a support structure such that the bottom membrane, top membrane and the support structure form an enclosed inner space not accessible by the immune system when implanted, and wherein the bottom membrane and the top membrane comprise a plurality of pores extending through the membrane to allow diffusion of nutrients and oxygen to the inner space, and wherein the device further comprises a mesh positioned in the inner space of the device such as to provide a support for the cells or cell aggregates when the cells or cell aggregates are inserted in the inner space of the device, such that the cells do not clump, wherein the mesh has a pore dimension between 60 and 300 μm.

In a second aspect, the invention relates to implantable device according to any one of the first aspect of the invention for use as a medicament, wherein the implantable device comprises cells or cell aggregates.

In a third aspect, the invention relates to a method of constructing the implantable device as defined in the first aspect of the invention, the method comprising: providing a bottom membrane; positioning a support structure roughly along the edges of the bottom membrane such as to leave a central space; positioning a mesh in the central space on top of the bottom membrane; positioning a top membrane on top of the support structure such as to cover the central space comprising the mesh as to completely enclose it creating an inner space; and fixing the top and bottom membrane to the support structure, preferably by ultrasonic welding, heat sealing, pressure sealing, adhesives or more preferably by ultrasonic welding.

In a fourth aspect the invention relates to a method for loading an implantable device with cells or cell aggregates, the method comprising: providing the device according to the first aspect of the invention or obtained or obtainable by the method of the second aspect of the invention, providing a cell or cell aggregate suspension in a container; connecting a tube such that one end is in open contact with the interior of the container with the cell or cell aggregate suspension, and that the other end is inserted through an opening into the inner space of the implantable device; and allowing the cells or cell aggregates in the container to drain through the tube into the inner space of the implantable device; and optionally closing the opening in the implantable device after removing the tube, preferably by ultrasonic welding, heat sealing, pressure sealing, adhesives or more preferably by heat sealing.

### Definitions

For purposes of the present invention, the following terms are defined below.

As used herein, the singular form terms “A,” “an,” and “the” include plural referents unless the content clearly dictates otherwise. Thus, for example, reference to “a cell” includes a combination of two or more cells, and the like. When used herein the term “cell” or “cells” specifically also includes “a cell aggregate” or “cell aggregates”.

As used herein, the term “and/or” refers to a situation wherein one or more of the stated cases may occur, alone or in combination with at least one of the stated cases, up to with all of the stated cases.

As used herein, the term “at least” a particular value means that particular value or more. For example, “at least 2” is understood to be the same as “2 or more” i.e., 2, 3, 4, 5, 6, 7, 8, 9, 10, 11, 12, 13, 14, 15, . . . , etc. As used herein, the term “at most” a particular value means that particular value or less. For example, “at most 5” is understood to be the same as “5 or less” i.e., 5, 4, 3, . . . −10, −11, etc.

As used herein, the word “comprise” or variations thereof such as “comprises” or “comprising” will be understood to include a stated element, integer or step, or group of elements, integers or steps, but not to exclude any other element, integer or steps, or groups of elements, integers or steps. The verb “comprising” includes the verbs “essentially consisting of” and “consisting of”.

As used herein, the term “conventional techniques” refers to a situation wherein the methods of carrying out the conventional techniques used in methods of the invention will be evident to the skilled worker. The practice of conventional techniques in molecular biology, biochemistry, computational chemistry, cell culture, tissue engineering, regenerative medicine, recombinant DNA, bioinformatics, genomics, sequencing and related fields are well-known to those of skill in the art and are discussed, for example, in the following literature references: Sambrook et al., Molecular Cloning. A Laboratory Manual, 2nd Edition, Cold Spring Harbor Laboratory Press, Cold Spring Harbor, N. Y., 1989; Ausubel et al., Current Protocols in Molecular Biology, John Wiley & Sons, New York, 1987 and periodic updates; and the series Methods in Enzymology, Academic Press, San Diego; Mozafari et al. Handbook of Tissue Engineering Scaffolds: Volume Two, 2019 Elsevier; Atala et al., Principles of Regenerative Medicine, Third Edition, 2019

As used herein, the term “in vitro” refers to experimentation or measurements conducted using components of an organism that have been isolated from their natural conditions.

As used herein, the term “subject” or “individual” or “animal” or “patient” or “mammal,” used interchangeably, refer to any subject, particularly a mammalian subject, for whom diagnosis, prognosis, or therapy is desired. Mammalian subjects include humans, domestic animals, farm animals, and zoo-, sports-, or pet-animals such as dogs, cats, guinea pigs, rabbits, rats, mice, horses, cattle, cows, bears, and so on. As defined herein a subject may be alive or dead. Samples can be taken from a subject post-mortem, i.e., after death, and/or samples can be taken from a living subject.

As used herein, terms “treatment”, “treating”, “palliating”, “alleviating” or “ameliorating”, used interchangeably, refer to an approach for obtaining beneficial or desired results including, but not limited to, therapeutic benefit. By therapeutic benefit is meant eradication or amelioration or reduction (or delay) of progress of the underlying disease being treated. Also, a therapeutic benefit is achieved with the eradication or amelioration or reduction (or delay) of progress of one or more of the physiological symptoms associated with the underlying disease such that an improvement or slowing down or reduction of decline is observed in the patient, notwithstanding that the patient can still be afflicted with the underlying disease.

As used herein, the term “implantable cell delivery device” is interchangeably used with “implant device”, “cell delivery device”, “implantable device”, “macro-encapsulating implant”, “carrier”, “cell carrier” or simply “device” and refers to an enclosure suitable for retaining cells and which enclosure is intended for implanting in a subject. The device thus serves as a vehicle to implant cells in a subject. Therefore, it may be assumed that the device is of a material suitable for implanting in a subject and that the device is constructed such that it is suitable to contain living cells.

As used herein the term “closed” when referring to the implantable cell delivery device implies that the device has, after sealing the cells inside, no openings large enough to allow intrusion of the device by immune cells or vascularization of the host.

The term closed does thus not necessarily exclude the presence of pores or openings that are present to allow mass transport of essential molecules, for example nutrients, gases, small proteins or peptides, metabolites, cytokines, hormones, and other biomolecules. When used herein, the term pore refers to an opening with a diameter which allows diffusion (or mass transport) of nutrients, gases, small proteins or peptides, metabolites, cytokines, hormones and other biomolecules but which diameter is too small to allow vascularization inside the device or to allow entry of an immune cell into the device.

When used herein, “pore size”, “pore dimension” or “mesh size”, when referring to a mesh refers to the size of the openings of the mesh and should be interpreted as the average diameter of the openings in case the openings are substantially circular, or the average size of the side when the openings are substantially square or rectangular.

### DETAILED DESCRIPTION OF THE INVENTION

The section headings as used herein are for organizational purposes only and are not to be construed as limiting the subject matter described.

A portion of this invention contains material that is subject to copyright protection (such as, but not limited to, diagrams, device photographs, or any other aspects of this submission for which copyright protection is or may be available in any jurisdiction). The copyright owner has no objection to the facsimile reproduction by anyone of the patent document or patent invention, as it appears in the Patent Office patent file or records, but otherwise reserves all copyright rights whatsoever.

Various terms relating to the methods, compositions, uses and other aspects of the present invention are used throughout the specification and claim. Such terms are to be given their ordinary meaning in the art to which the invention relates, unless otherwise indicated. Other specifically defined terms are to be construed in a manner consistent with the definition as provided herein. The preferred materials and methods are described herein, although any methods and materials similar or equivalent to those described herein can be used in the practice for testing of the present invention.

Unless otherwise defined, all technical and scientific terms used herein have the same meaning as commonly understood by a person of ordinary skill in the art.

Present invention relates to an immunoprotective type implantable cell delivery devices, or closed devices, intended to implant cells in a subject, for example as a means of treating a disease. Exemplary cells that could be used in implantation therapy are islet cells in the treatment of diabetes, but other cells are known to the skilled artisan which could be used in therapeutic methods by implantation.

Encapsulating cells, such as islet cells, prior to transplantation can potentially overcome the challenges faced in the current clinical cell transplantation therapy (such as clinical islet transplantation). It is appreciated however that the device can be used for any type of treatment that benefits from transplantation of cells or cell aggregates, including novel therapies for example based on induced pluripotent stem cell or trans-differentiation derived cell sources. Here, the inventors describe the development of an7mmuneprotective islet encapsulation device made of porous membranes, which protects cells from the immune cells and keeps cells viable and functional by allowing nutrient diffusion. There are three main challenges in the current development of cell encapsulation devices. First, by avoiding or having a reduced foreign body response by the selection of a biocompatible material, the inventors screened potential biomaterials and based on these results selected polyvinylidene fluoride (PVDF) as an exemplary candidate biocompatible material. Second, by enabling the transport of nutrients, gases, small proteins or peptides, metabolites, cytokines, hormones and other biomolecules to the encapsulated cells, by the production of thin and porous membranes and finally protect the encapsulated cells against the immune system by using immunoprotective membranes with pores that do not allow passage of immune cells.

Third, a common problem is that cells enclosed in the implant device tend to clump together forming large aggregates which become necrotic in the interior due to lack of oxygen and nutrients. This problem has been addressed by for example using hydrogels in the lumen of the device to embed the cells or cell clusters. Although this solution prevents cell clumping to large aggregates to some degree, it was found that the presence of a hydrogel still interferes with diffusion of oxygen and nutrients, and also prevents factors produced by the cells (e.g. insulin) to effectively diffuse out of the device.

When used herein, the terms membrane and film or thin film are used interchangeable and refer to a flat sheet like material.

Therefore, in a first aspect the invention relates to an implantable device of the immunoprotective type for implanting cells or cell aggregates in a subject, wherein the device comprises a bottom membrane and a top membrane, wherein the bottom membrane and the top membrane are joined together with a support structure such that the bottom membrane, top membrane and the support structure form an enclosed inner space not accessible by the immune system when implanted, and wherein the bottom membrane and the top membrane comprise a plurality of pores extending through the membrane to allow diffusion of nutrients and oxygen to the inner space, and wherein the device further comprises a mesh positioned in the inner space of the device such as to provide a support for cells when the cells are comprised in the inner space of the device, such that the cells do not clump, wherein the mesh has a pore dimension between 60 and 300 μm. Thus, inclusion of the mesh prevents or reduces the formation of large clumps of cells, resulting in the cells or cell aggregates to survive longer and function better. When used herein the term clump or to clump refers to a large cell aggregate (or formation thereof) where the cell aggregate experiences a negative effect due to its large size, such as but not limited to necrosis, impaired growth or function.

It was found that using a mesh could effectively address the problem of cell clumping. Although meshes have been described in open type cell delivery devices (see e.g. US 2017/182139), the inventors are not aware of similar solutions in a closed (immunoprotective) device. The reason for this is that such devices are very hard to properly populate with cells or cell aggregates. Such devices are preferably assembled and provided without cells, and seeded with cells upon use, just prior to implanting the device in a subject. However, it is challenging to seed the cells in a closed device through a narrow opening and enable even distribution of the cells (or cell clusters) over the mesh in the device, generally the cells will end up as a massive clump in the device.

Theoretically the device can undergo a final assembly prior to implanting, meaning that cells are seeded on a mesh which is then enclosed between the outer layers of the device (membranes), allowing even distribution of the cells on the mesh. In practice however this solution does not work as assembling and securing the components together is too disruptive for the cells, and either causes cells to die or to be displaced in the device. The inventors have now developed a new method of seeding cells in the device as described below, allowing the evenly seeding of cells (or cell aggregates) in the already assembled device, thus allowing a practical solution for a closed device with a mesh.

As is of concern with any transplantation site or implantation, proper nutrient diffusion is essential to ensure optimal survival and functioning of the transplanted graft. For example, pancreatic islets show a high metabolic activity and therefore require swift access to oxygen and nutrients to survive. It is therefore vital that the delivery devices are as thin and porous as possible to reduce the diffusion pathway length and minimize mass transport time to and from the cells inside the implant. The present invention therefore provides, among others, methods to fabricate an immunoprotective cell delivery device to realize cell delivery, and to allow implant design having clinically relevant device dimensions.

The implantable cell delivery device according to the invention is intended to implant cells in a subject. The examples shown in this patent illustrate that the delivery device is not limited to only beta cells and islets, but can be used for cell delivery in general such as human mesenchymal stem cells, alpha cells, or any other relevant cell type. Based on the examples it is understood that other cell types, mixtures of cell types, organoids or (parts of) tissue or organs can be included in the implantable cell delivery device. As the device is intended for implantation in a subject, there are certain limitations to e.g. the materials used which must be biocompatible as well as the dimensions of the device. It is understood that the dimensions may depend on the subject in which the device is intended to be implanted. When used herein, the term subject may refer to an animal such as a rodent or a mammal or a human. Therefore, the size limitations for an implantable cell delivery device are different for e.g. a mouse when compared to a human, however even within the same species differences in e.g. body size may influence the size of the implantable cell delivery device. The size of the device is further influenced by the cell type intended to be included in the implantable cell delivery device, and the function or purpose of implanting the cells. The skilled person is capable to estimate an approximate desired size of the implant based on, among others, the subject and the cell type to be implanted.

The implantable cell delivery device according to the invention comprises a bottom membrane and a top membrane, wherein the bottom membrane and the top membrane are joined together with a support structure such that the bottom membrane, top membrane and the support structure form an enclosed inner space not accessible by the immune system when implanted, wherein the bottom membrane and the top membrane comprise a plurality of pores extending through the membrane to allow diffusion of nutrients and oxygen to the inner space. The device further comprises a mesh positioned in the inner space of the device. The mesh is intended as a support and spacer structure for the cells. It was found by the inventors that using a mesh effectively prevented aggregation of cells or cell clusters both in cell culture and when used in a device (see e.g. FIG. 12). Aggregation of cells or cell clusters is not desirable and a problem with presently known cell implant devices. Aggregation of cells or cell clusters leads to too large cell aggregates, where the inner cells are deprived of nutrients and oxygen, resulting in necrosis, loss of function, potency and efficacy, among others. Therefore, additional means are needed to keep cells or cell clusters separated. This patent describes a method to prevent cells from aggregating inside the cell delivery device by using a mesh, which advantageously allows easy diffusion of nutrients, while cells and cell aggregate are kept separated. Both the top membrane and the bottom membrane have a plurality of pores allowing the diffusion of nutrients and oxygen towards the cells, and optionally, secreted factors from the cells out of the device. A non-limiting example of a secreted factor is insulin.

When used herein, a membrane refers to a thin and flat material. When used herein, the surface area of the membrane refers to upper and lower surfaces wherein the upper and lower surfaces are those surfaces opposing each other with the lowest thickness in between. When used herein a pore refers to an opening or cavity in the membrane that completely penetrates the membrane and thus allows for the passage of e.g. molecules from one side of the membrane to the other, preferably from upper to the lower surfaces.

The implantable cell delivery device according to the invention further comprises a supporting structure positioned substantially around the surface area of the bottom membrane and the surface area of the top membrane such that the supporting structure is positioned in the plane of the surface areas of the top and the bottom membranes. The supporting structure may for example be oval or round, but it is understood that it may have any kind of shape. Ideally, the shape of the supporting structure follows the contours of the top and bottom membranes. For example, when the bottom and top membrane have an oval shape, the supporting structure is preferably an oval shaped ring following the edges of the bottom and top membranes. It is understood that the supporting structure may have gaps or spaces, for example the supporting structure may also be U-shaped.

It is understood that the device may comprise additional supporting structures. For example, the immunoprotective type implantable cell delivery device according to the invention may comprise one or more additional support structures, preferably wherein said one or more additional support structured are positioned more centrally with respect to the bottom and top membranes.

The function of the supporting structure is to provide some rigidity to the device. Although some degree of flexibility is desirable in an implantable cell delivery device, the structural integrity must be ensured. Because the bottom and top membranes must allow the diffusion of nutrients and oxygen, there are limitations to the thickness of the membranes which in general are very thin and thus fragile. The supporting structure helps to avoid tearing or rupturing of the membranes. Further, inclusion of the supporting structure prevents folding or bending of the device, which could otherwise lead to an increased inner space, resulting in an increased diffusion path and reduced function of the cells, which is undesirable for the recipient of the device. Therefore, the supporting structure has a thickness which is generally more than the thickness of the bottom and top membranes. For example, the thickness of the top and bottom membranes may each individually be between 10 and 250 μm thick, preferably between 35 and 200 μm more preferably between 40 and 160 μm, while the supporting structure may be around 75 to 500 μm thick, preferably between 100 to 400 μm more such as for example around 200 μm thick.

The supporting structure further provides a scaffold for attaching the bottom and top membranes. The membranes may be attached to the supporting structure such that the supporting structure is sandwiched between the edges of the membranes, alternatively the membranes may be attached together on one side of the supporting structure, e.g. the top or the bottom side. The membranes may be attached for example by ultrasonic welding, heat sealing pressure sealing or adhesives, but ultrasonic welding is preferred.

The implantable cell delivery device according to the invention further has the bottom and the top membrane attached to the support structure in one or more places such as to substantially, preferably completely, seal of the inner space when being implanted. Therefore, the device when ready to implant only allows diffusion of molecules such as nutrients and oxygen through the pores, therefore the nutrient diffusion can be controlled through controlling the pore size of the membranes. It is understood however that cells need to be loaded in the device, therefore the device is constructed to leave an opening between the top and/or bottom membrane and the support structure such to allow cells to be inserted in the device as described below. After cells have been inserted in the device, the opening is to be sealed such that nutrient diffusion can only take place through pores thus creating a barrier for immune cells that allows nutrient diffusion. Therefore, when used herein a pore should be interpreted as an opening in the devices that due to its size does not allow passage of immune cells into the device.

It was found that the device is preferably constructed from PVDF, as it has improved porosity compared to other suitable materials while maintaining mechanical strength. A further advantage is that PVDF is biocompatible, and thus has a much reduced immune response, meaning the cells in the device are less affected due to the strongly reduced immune response compared to devices that are not biocompatible and therefore display a much stronger immune response. It is understood that the PVDF may be combined with a suitable material, a non-limiting example being polyvinylpyrrolidone (PVP).

Therefore, in an embodiment the bottom membrane and/or the top membrane and optionally the support structure are made from a non-water soluble polymer, preferable of polyvinylidene fluoride (PVDF), polyethersulfone (PES), poly(ethylene oxide terephthalate)/poly(butylene terephthalate) (PEOT/PBT) or a non-water soluble polymer mixed with a water soluble co-polymer, preferably wherein the membranes further comprise a pore forming reagent, more preferably wherein the pore forming reagent is polyvinylpyrrolidone (PVP).

An additional advantage of using PVDF, as the material to manufacture the device, is that it allows spot welding. Therefore, in an embodiment the top and bottom membrane are attached to the support structure by spot welding. Spot welding has the advantage that no additional materials need to be used such as a glue, which may trigger an immune reaction, may not be biocompatible or even toxic, or may dissolve over time resulting in structural failure of the device.

It is further envisioned that a marker for imaging is included in or on the device. This may be advantageous as it allows for locating the device in a subject without the need for surgical procedures. Therefore, in an embodiment the device comprises one or more markers for imaging, preferably wherein said one or more markers comprise a radiopacifier infused in or coated on the PVDF of the top membrane, the bottom membrane and/or the support structure, more preferably wherein said radiopacifier is barium based such as barium sulfate, bismuth based such as bismuth trioxide, bismuth subcarbonate or bismuth oxychloride, or wherein the radiopacifier is tungsten or graphene oxide.

When used herein, a radiopacifier, also referred to as radiocontrast material, is a substance that is opaque for the radio- and x-ray waves portion of the electromagnetic spectrum, meaning a relative inability of those kinds of electromagnetic radiation to pass through the particular material. Non-limiting examples of radiocontrast materials include titanium, tungsten, barium sulfate, bismuth oxide and zirconium oxide. Some solutions involve direct binding of heavy elements, for instance iodine, to polymeric chains in order to obtain a more homogeneous material which has lower interface criticalities.

It is further envisioned that it may be advantageous to include a drug or compound with the device. Therefore, in an embodiment the device comprises a drug or compound infused in or coated onto the PVDF membrane, including the top and bottom membrane and/or the support structure. For example, the device may be coated with a drug to prevent fibrous capsule forming or a drug to regulate the foreign body response. Alternatively, a therapeutic drug may be included as a co-treatment in case the device is implanted as a treatment option in the subject. Non-limiting examples are chemotherapeutical agents for treatment of cancer, immune checkpoint inhibitors, cell stress inhibitors aiding in cell cluster and/or organoid survival in the early post-surgery period, or imaging markers for tracking the implant post-surgery. Further envisioned is the inclusion of angiogenic factors to promote vascular ingrowth in the device.

The pores in the top and bottom membranes allow diffusion of nutrients and oxygen to the cells in the device. Ideally the average pore size is chosen such to allow maximal diffusion while maintaining structural integrity and preventing the cells from exiting the device. In an embodiment, the pores are created by including a soluble polymer in the film, which allows the creation of pores in the film. Therefore, in an embodiment the (average) pore size of the bottom membrane and the top membrane is between 10 and 500 nm, preferably between 50 and 450 nm more preferably between 100 and 400 nm. The pores size may be varied for example by controlling the ratio of (water) soluble to non-soluble polymer used during film manufacturing. It is understood that other methods can be used which are known to the person skilled in the field Further, it is understood that the pore size is limited by immune cells, as the pores should not allow immune cells to enter the device. The average pore size in the top and the bottom membrane may be the same or may be different.

It is envisioned that the thickness of the membranes is a balance between structural integrity and ability to diffuse nutrients, where a thicker membrane is stronger but allows less diffusion and vice versa. Therefore, in an embodiment the bottom membrane and the top membrane have a thickness of between 10 and 200 nm, preferably between 35 and 200 nm, more preferably between 40 and 160 nm. It was found that these values provide a good balance between strength and diffusion ability.

It was found that the mesh can be constructed from any non-degradable polymer, however biocompatible polymers are preferred. Therefore, in an embodiment the mesh is constructed from a non-degradable polymer, preferably wherein the non-degradable polymer is selected from nylon, PVDF, PTFE, PP, PE, or PET. In an embodiment the mesh has a pore dimension of between 50 and 300 μm, preferably between 60 and 200 μm, more preferably between 70 and 150 μm, most preferably between 75 and 120 μm to accommodate islet cells, however it is understood that for different cell types or organoids a mesh with different pore dimensions are desirable. The person skilled in the art is able to determine a suitable pore dimensions for the respective cell type or organoid.

In an embodiment the device comprises cells in the inner space, preferably wherein the cells are cell aggregates and/or organoids, preferable wherein the cell aggregates are endocrine cells or cytokine producing cells or aggregates thereof, preferably wherein the cell aggregates are selected from islet cells, human mesenchymal stem cells, kidney cells, thyroid cells, thymic cells, testicular cells, pancreatic cells, endocrine cells, liver cells, cytokine producing cells, or preferably wherein the organoid is selected from a kidney organoid, an intestinal organoid, a pancreatic organoid, a neural organoid, a hepatic organoid, a thyroid organoid, a stomach organoid, an ovarian organoid, a prostate organoid, a splenic organoid, oesophageal organoid, a breast organoid, a bladder organoid, a lung organoid, an optic organoid, an inner ear organoid, a cardiac organoid, a biliary organoid, a salivary gland organoid, a pituitary gland organoid, a lymphoid organoid, a bladder organoid, a tongue organoid, a cerebral/brain organoid, a spinal cord organoid, a fallopian tube organoid, a lacrimal gland organoid, a skin organoid, a thymic organoid, a testicular organoid, an epithelial organoid, a gastruloid (embryonic organoid), a blastoid (blastocyst-like organoid), a retinal organoid and a hippocampal organoid. The cell aggregates may also refer to a resected piece of tissue or organ, for example obtained from a donor organism.

It is understood that the size of device can be scaled depending on the intended application (e.g. treatment method or type of cells contained in the device) and based on the subject. It will be clear to the skilled person that a device intended for implantation in a human subject needs to be larger than a device intended to be implanted in a rodent. Because the device is essentially two-dimensional, meaning existing of a single plane with wells, it is anticipated to for some applications the device needs to be scaled to an impractical size in larger mammals such as humans. It is therefore further envisioned that multiple smaller versions of the device can be stacked together or implanted separately in the subject. Therefore, in an embodiment, the invention relates to an implantable device according to the first aspect of the invention, wherein the device comprises two or more implantable devices stacked together and separated by a spacer such as to create an open space between the individual stacked implantable devices. The additional support structure essentially functions as a spacer between the individual devices.

Two or more devices can in principle be stacked to obtain a more compact design, as long as proper diffusion of nutrients and oxygen to the cells is ensured. This becomes particularly relevant when three or more layers are stacked with respect to the middle layers (devices). It is therefore found by the inventors that stacked layers (devices) should be separated by a spacer. Preferably the spacer is constructed such that the space between layers is not completely enclosed by the spacer, allowing east diffusion of molecules between the devices. Therefore, either several small spacers may be used, or the spacer may have openings. The spacer may be regarded as an additional support structure, therefore when used herein the spacer may also be referred to as “additional support structure”. Preferably, the spacer is also constructed from PVDF to ensure biocompatibility. It is further envisioned that the support structures and the spacer(s) (additional support structure) are one continuous structure. The spacer may be attached to the support structure or the membranes using ultrasonic welding, heat sealing, pressure sealing, or adhesives, more preferable by ultrasonic welding.

It is envisioned that the device may be used in a medical method or a method of treatment. Therefore, in a second aspect the invention relates to the immunoprotective type implantable cell delivery device according to the invention for use as a medicament. The invention further relates to the immunoprotective type implantable device according to the first aspect of the invention for use in the treatment, prevention or amelioration of a disease. The device preferably comprises cells, more preferably cell aggregates or organoids, therefore, in an embodiment the invention relates to the immunoprotective type implantable cell delivery device comprising cells, preferably cell aggregates and/or organoids, according to the invention for use in the treatment, prevention or amelioration of a disease. Alternatively, the invention relates to a method of treating, preventing or ameliorating a disease or a condition in a subject in need thereof, the method comprising implanting the device comprising cells, preferably cell aggregates or organoids, in the subject.

Several treatment options are envisioned for the device. For example, the device may be used in the treatment of diabetes. Therefore, in an embodiment, the treatment is treatment of diabetes, preferably type 1 diabetes. Preferably when the treatment is treatment of diabetes the device comprises insulin secreting cells, such as islet cells or cells engineered to secrete insulin.

It will however be clear to the skilled person that use of the device is not limited to treatment of diabetes, as the device allows for incorporation of any type of cell, cell aggregate or organoid. The device may for example be used for implanting cells that excrete an extracellular factor which may be beneficial in a therapy. Non-limiting examples of extracellular factors include peptides and proteins such as insulin, glucagon, cytokines, growth factors, hormones, carbohydrates, and clotting factors.

Therefore the device may be used, but not limited to, for treatment of immune related disorders such as multiple myeloma, melanoma, rheumatoid arthritis, inflammatory bowel disease, lupus, scleroderma, hemolytic anemia, vasculitis, type 1 diabetes, Graves' disease, multiple sclerosis, Goodpasture syndrome, pernicious anemia, myopathy, Lyme disease, severe combined immunodeficiency (SCID), DiGeorge syndrome, hyperimmunoglobulin E syndrome (also known as Job's Syndrome), common variable immunodeficiency (CVID), chronic granulomatous disease (CGD), Wiskott-Aldrich syndrome (WAS), autoimmune lymphoproliferative syndrome (ALPS), hyper IgM syndrome, leukocyte adhesion deficiency (LAD), NF-κB essential modifier (NEMO) mutations, selective immunoglobulin A deficiency, X-linked agammaglobulinemia (XLA; also known as Bruton type agammaglobulinemia), X-linked lymphoproliferative disease (XLP), Ataxia-telangiectasia or Acquired immunodeficiency syndrome (AIDS). Further, the device may be used in the treatment of a growth factor related disease such as cancer. Further, the device may be used in a hormone or endocrine related disorder such as adrenal insufficiency, Addison's disease, Cushing's disease, Cushing's syndrome, gigantism (acromegaly), hyperthyroidism, Grave's disease, hypothyroidism, hypopituitarism, multiple endocrine neoplasia I and II (MEN I and MEN II), polycystic ovary syndrome (PCOS) or precocious puberty. Further, the device may be used for the treatment of a clotting disorder such as Factor V Leiden, prothrombin gene mutation, deficiencies of natural proteins that prevent clotting (such as antithrombin, protein C and protein S), elevated levels of homocysteine, elevated levels of fibrinogen or dysfunctional fibrinogen (dysfibrinogenemia), elevated levels of factor VIII and other factors including factor IX and XI, abnormal fibrinolytic system, including hypoplasminogenemia, dysplasminogenemia and elevation in levels of plasminogen activator inhibitor (PAI-1) cancer, obesity, pregnancy, supplemental estrogen use, including oral contraceptive pills (birth control pills), hormone replacement therapy, prolonged bed rest or immobility, heart attack, congestive heart failure, stroke and other illnesses that lead to decreased activity, heparin-induced thrombocytopenia, antiphospholipid antibody syndrome, previous history of deep vein thrombosis or pulmonary embolism, myeloproliferative disorders such as polycythemia vera or essential thrombocytosis, paroxysmal nocturnal hemoglobinuria, inflammatory bowel syndrome, HIV/AIDS or nephrotic syndrome. Further, the device may be used in a regenerative medicine indication such as but not limited to spinal cord injuries, type 1 diabetes, Parkinson's disease, amyotrophic lateral sclerosis, Alzheimer's disease, heart disease, stroke, burns, cancer and osteoarthritis.

In a third aspect the invention relates to a method of constructing the implantable device as defined in any one of claim 1, the method comprising: providing a bottom membrane; positioning a support structure roughly along the edges of the bottom membrane such as to leave a central space; positioning a mesh in the central space on top of the bottom membrane; positioning a top membrane on top of the support structure such as to cover the central space comprising the mesh as to completely enclose it creating an inner space; and fixing the top and bottom membrane to the support structure, preferably by ultrasonic welding, heat sealing, pressure sealing, or adhesives, more preferable by ultrasonic welding. In an embodiment the top and bottom membranes are fixed to the support structure such as to leave a single opening for loading the device with cells.

When used herein, the term “spot welding” preferably refers to ultrasonic spot welding. Ultrasonic spot welding is an industrial process whereby high-frequency ultrasonic acoustic vibrations are locally applied to workpieces being held together under pressure to create a solid-state weld. It is commonly used for plastics.

The inventors are not aware of any other immunoprotective type implantable cell delivery device, which comprises a mesh. A reason may be that there are several obstacles for the construction of such device such as that it is not straightforward how the cells can be loaded in the device. One method would be to seed the cells in the bottom layer prior to assemble of the device, however there are several impracticalities, as the device is preferably shipped in assembled form to the surgeon or researcher who will use the device or the patient or subject that will receive the device. Moreover, welding polymer layers near cells will lead to severe loss of cell viability. To overcome these issues, the inventors have developed a method of seeding the device which does not require assemble of the device after seeding, meaning the device is seeded when completely assembled. Therefore, in a fourth aspect the invention relates to a method for loading an implantable device with cells, the method comprising: providing the device according to the first aspect of the invention or obtained or obtainable by the method of the second aspect of the invention, providing a cell suspension in a container; connecting a tube such that one end is in open contact with the interior of the container with the cell suspension, and that the other end is inserted through an opening into the inner space of the implantable device; and allowing the cells in the container to drain through the tube into the inner space of the implantable device; and optionally closing the opening in the implantable device after removing the tube, preferably by ultrasonic welding, heat sealing, pressure sealing, or adhesives, more preferable by heat sealing.

The device upon construction is closed completely except for the seeding inlet. Cells will be seeded through the seeding inlet by holding the device almost horizontally. A tube with the cell suspensions is inserted into the single opening in the interior of the devices between the top membrane and the mesh. In this way the cells, cell clusters or organoids are captured inside the mesh structure.

Therefore, in an embodiment the cells are allowed to migrate through the tube into the inner space of the immunoprotective type implantable cell delivery device by gravity.

### BRIEF DESCRIPTION OF THE FIGURES

FIG. 1: Representative SEM images of surface morphology and cross section of produced PVDF and PVDF/PVP membranes. 15% (w/w) PVDF membrane casted at 200 μm (A-C) and 15% (w/w) PVDF+5% (w/w) PVDF membranes casted at 200 μm (D-F). (A-F) Views of the glass side at 5.000× and 25.000× magnification (A, D); air side at 5.000× and 25.000× magnification (B, E); and cross section (C, F). Scale bar represents 4 μm in A, B, D and E. Scale bar represents 50 μm in C and F. (G) The thickness of produced membranes measured from SEM cross-section images. (H) Pore diameter of produced membranes measured at the air-exposed site. Data presented as mean±SD, n=3. * P<0.05, with t-test.

FIG. 2: Physical properties of the produced PVDF-alone and PVDF/PVP membranes. (A) Water contact angle from both sides of the produced membranes. Data presented as mean±SD, n=9. * P<0.05, with student t-test. (B) Representative images of water contact angle measurements of the produced membranes. (C—F) Mechanical properties of produced membranes characterized by the Young's modulus (C), peak stress (D), failure strain I, and failure stress (F). Data presented as mean±SD, n=5. * P<0.05, with student t-test.

FIG. 3: Indirect cytotoxicity tests and chemical composition of the produced PVDF-alone and PVDF/PVP membranes. (A) Levels of metabolic activity of INS-1E cells in contact with different exposure media during a MTT assay. Data presented as mean±SD, N=3, *P<0.05, one-way ANOVA. (B) Measurement of LDH release of INS-1E cells in contact with different exposure media. Data presented as mean±SD, N=3, *P<0.05, one-way ANOVA. (C) FTIR spectra of produced membranes. The green line at ˜1660 cm⁻¹ indicates a C═O peak present in PVDF/PVP membranes. The red and black lines at ˜1400 and ˜1180 cm⁻¹ respectively correspond to CH2 and CF2 stretching groups present in the PVDF base structure.

FIG. 4: Diffusion properties of different molecules over the produced PVDF-alone and PVDF/PVP membranes, either in 2-chamber diffusion setup (A-J) or in assembled devices (K-L). (A-C) Diffusion of 2 mg/ml of 3-5 kDa fluorescently labelled dextran beads over 200 min (A), its calculated flux (B) and diffusion coefficient (C). Data presented as mean±STD, n=5. * P<0.05, with student t-test. (D-F) Diffusion of 20 mM of D-glucose over 200 min (D), its calculated fll(E) and diffusion coefficient (F). Data presented as mean±STD, n=5. * P<0.05, with student t-test. (G-I) Diffusion of FITC-labelled insulin, as a percentage of total after 24 h (G), its calculated flux (H), and diffusion coefficient (I). Data presented as mean±STD, n=5. * P<0.05, with student t-test. (J) Diffusion of IgG, as a percentage of total after 3 h. Data presented as mean±STD, n=4. (K-L) Diffusion of oxygen from 21% oxygen towards 5% oxygen (K) and area under the curve (AUC) of oxygen diffusion (L). Data presented as mean±STD, N=3.

FIG. 5: Immunoprotective properties of produced membranes, PVDF and PVDF/PVP. (A) Representative SEM images of fixed human primary macrophages seeded on top of different porous transwell inserts (0.4, 1, 3 and 8 μm pores) or produced membranes. Cells that were able to migrate through the pores can be seen in the representative bottom image. Scale bar represents 10 μm in all images (B) Quantification of migrated human primary macrophages on the bottom of 24-well plate. Data presented as mean±STD, N=3. (C) Representative light microscopy images of well plate bottoms of 3 and 8 μm transwell after 24 h. Scale bar represents 200 μm.

FIG. 6: Schematic illustration of device production and islet seeding. (A) Three layers of the device, two membrane sheets (light gray) and the support ring (dark gray) in between. (B) Sealing the three layers of the device with ultrasonic welding. (C) Assembled device with a seeding inlet. (D) Islet seeding through the seeding ilt. (E) Closing the seeding inlet with heat sealing. (F) Culture of the cell loaded device.

FIG. 7: Production and testing of encapsulation devices. (A) Schematic illustration of the assembly of the encapsulation device. (B) Assembled encapsulation device. (C) Handling assembled encapsulation device. Camera shots of device testing for leakage (D-E). (D) Leakage free device and leakinlevice (E). Mechanical properties of heat and ultrasonic welded sea (F-G). (F) Failure stress and (G) failure strain of seals. Data presented as mean±std, n=5. * P<0.05, with student t-test.

FIG. 8: Human islets cultured inside the immunoprotective device. (A) Representative images of human islets inside the encapsulation devices after 8 days of culturing. Red channel represents dead cells, green channel represents live cells. Scale bar represents 200 μm in all pictures. (B) Quantification of human islet viability after 8 days of culturing inside the encapsulation devices or without devices as control. Data presented as mean±STD, N=3. (C) Insulin release profile of human islets during GSIS after 8 days of culturing inside the devices normalized against total insulin. * P <0.05, with one way ANOVA. (D) Stimulation index at day 8. Data presented as mean SEM, N=3. * P<0.05, with Kruskal-Wallis test.

FIG. 9: schematic overview of different mesh sizes. The figure schematically depicts different mesh sizes, the indicated sizes in micrometer are indicative for islet cells, other cell types or organoids may requires differently sized meshes. Depicted are schematically the situations (top: top view; bottom: cross sectional view) of the situation where the mesh is too small (left) and cells may still cluster, ideal mesh size (middle) where cells are isolated from each other or where the mesh is too large (right) where cells fall through the mesh and may still cluster.

FIG. 10: schematic cross section of the device. Depicted are the top and bottom membranes, the mesh and cells inside the device embedded on the mesh. Further shown is the support structure, in this case a support ring.

FIG. 11: shows cross sections of a stacked device. Depicted are two device stacked together and separated by additional support structures to allow diffusion of nutrients.

FIG. 12: Islet cells cultured on a mesh do not aggregate. Pancreatic islet cells cultured in medium in a tissue culture dish, in the absence of a mesh (left) or with mesh (right).

FIG. 13: Islet cells cultured on a mesh do not aggregate. Pancreatic islet cells cultured inside an implant device as described herein, in the absence of a mesh (left) or with mesh (right).

FIG. 14: hMSC cell clusters can be cultured in the device. Depicts clusters of hMSC cells cultured outside or inside a device as described herein, depicted are viable cell clusters as determined by staining (not shown).

FIG. 15: alpha-TC cell clusters can be cultured in the device. Depicts clusters of alpha-TC cells cultured outside or inside a device as described herein, depicted are viable cell clusters as determined by staining (not shown).

FIG. 16: Seeding human islets inside devices with a mesh (pore size 400 μm) and devices without meshes

The top depict overview pictures of devices without (left) or with (right) a mesh after seeding of cells. A 400 μm pore size device was used. The bottom pictures depict a magnification showing individual cell clusters.

FIG. 17: Seeding beta cell aggregates into devices with ˜50 μm mesh The left picture depicts an overview of the device with a 50 μm mesh after seeding of cells. Right is a magnification depicting the individual cell clusters.

FIG. 18: Seeding pseudo-islets on top of a mesh with 80 μm pores A) Depicts cells grown for 4 days after seeding in a device without mesh in two different concentrations (left 1000 clusters per 2 mL, right 3000 clusters in 1.5 mL). B) Depicts cells grown for 4 days after seeding in a device with a 80 μm mesh in four different concentrations (from left to right: 1000 clusters per 1 mL, 1000 clusters per 2 mL, 1000 clusters per 5 mL, and 3000 clusters in 1.5 mL). Pictures are taken from bottom side of the mesh (top row) and top of the mesh (bottom row).

### EXAMPLES

### Example 1

### Materials and Methods

### Porous Membrane Production

Casting solutions were made by dissolving 15% (w/w) polyvinylidene fluoride (PVDF) (Kynar 720, Foster, Germany) in N,N-Dimethylacetamine (DMA) (Sigma-Aldrich, the Netherlands); and for polyvinylpyrrolidone (PVP) containing membranes, 5% (w/w) PVP (40 kDa) (Kollidon® 30, BTC, the Netherlands) was added. Solutions were mixed overnight at room temperature (RT) on a roller bank until a homogenous solution was obtained without air bubbles. Solutions were then casted on glass plates using an automatic film applicator (Elcometer, the Netherlands) equipped with a 200 μm casting knife (at RT and relative humidity of <30%), immediately followed by direct immersion of the glass plates into a coagulation bath (distilled water at RT). Membranes were collected from the coagulation bath and dried between paper tissues for two days before further use.

### Scanning Electron Microscopy (SEM)

Membrane samples were fixed on metal scanning electron microspy stubs using carbon tape and gold coated using a Cressington sputter coater, (Cressington 108 auto, United Kingdom). Cross-sections of membranes were made by immersing membranes in liquid nitrogen prior to breaking eah sample in half prior to imaging. Electron micrographs were taken using a scanning electron microscopy (SEM) (Teneo, FEI, the Netherlands) under high vacuum using secondary electron mode with ETD detection, 10 mm working distance and 5.00 kV acceleration voltage.

### Support Ring Production

PVDF pellets were preheated between a metal mold (200 μm thick, made in house) inside a hot press (Specac, United Kingdom) at 180° C. for 1 minute. Next, 20 kN was applied to soften pellets for 1 minute to create a solid film. Finally, the metal mold was removed from the hot press and cooled down for 5 minutes at RT before detaching the solid PVDF film from the mold.

### Membrane Wettability

Membranes were mounted horizontally on a glass slide. Droplets of 4 μL of distilled water (at RT) were dispensed on the surface of the membrane at a speed of 10 μL/min and high-resolution images were taken. Contact angles were measured by using Drop Shape Analysis 4 software equipped with a drop shape analyser (Krüss, Germany). Measurements of nine droplets were taken for each membrane.

### Mechanical Tensile Testing

Mechanical tensile properties of produced films and sealing methods were determined by mechanical tensile testing instrument (45 kN load cell, Electroforce 3230-ES Series III, United Kingdom). Dimensions of test samples were 35×10 mm, with an effective area of 15×10 mm between the clamps. The ramp speed was 0.05 mm/s. Each condition was tested five times. Obtained stress-strain curves were used to calculate peak strain, failure stress, failure strain and Young's modules. The different device components are annealed to each other using two different methods for comparison: heat sealing by a flat wire impulse sealer (Durapak, United States) and ultrasonic welding (LPX, Branson Ultrasonics, the Netherlands). Subsequently, the strength of the seals was determined with mechanical tensile.

### Cell Culture

INS-1E rat insulinoma cells (Addexbio Technology, United States) were cultured in Roswell Park Memorial Institute (RPMI) medium with L-glutamine (Sigma-Aldrich, the Netherlands) supplemented with 10% (v/v) Fetal Bovine Serum (FBS) (Sigma-Aldrich, the Netherlands), 10 mM HEPES (Thermo Fisher Scientific, the Netherlands), 1 mM sodium pyruvate (Thermo Fisher Scientific, the Netherlands), 5 mM glucose (Thermo Fisher Scientific, the Netherlands), 23.8 mM sodium bicarbonate (Thermo Fisher Scientific, the Netherlands) and 50 mM beta-mercaptoethanol (Thermo Fisher Scientific, the Netherlands). INS-1E P38 till P43 were used. Human primary macrophages (Celprogen, United States) were cultured′ in Dulbecco's Modified Eagle Medium (DMEM) high glucose (Thermo Fisher Scientific, the Netherlands) supplemented with 10% (v/v) FBS (Sigma-Aldrich, the Netherlands). Macrophages P6 till P10 were used. All cells were cultured at 37° C. and 5% CO₂.

### Human Islet Culture

Human islets were provided by the Human Islet Isolation Laboratory at Leiden University Medical Centre (LUMC, the Netherlands) which has permission from the Dutch government to isolate human islets with clinical intend. Organ donors (2 males and I donor gender unknown) had an average age of 43±14 years. The average islet purity was 75±5%. Islets were cultured in CMRL-1066 medium (Pan Biotech, Germany) supplemented with 10% (v/v) FBS (Sigma-Aldrich, the Netherlands), 10 mM HEPES (Thermo Fisher Scientific, the Netherlands), 1% Penicillin-Streptomycin (Thermo Fisher Scientific, the Netherlands) and 10 μg/mL Ciprofloxacin (Sigma-Aldrich, the Netherlands). Islets were cultured at 37° C. and 5% CO₂.

### MTT Assay

The cellular metabolic activity was assessed by measuring the conversion of 3-(4,5-dimethylthiazol-2-yl)-2,5-diphenyltetrazolium bromide (MTT) into formazan. This process only takes place in metabolic active cells. Extraction samples were generated by 24 h incubation of test materials in RPMI cell culture medium (see section cell culture). 1% Triton X-100 (VWR, the Netherlands) in RPMI was used as negative control, while untreated medium is used as positive control. INS-1E cells were seeded with 5×10⁵ cells/mL in 96-well plate (100 μL) and incubated overnight to form a 50% confluent monolayer in RPMI medium. After 24 h medium was removed and 100 μL extract sample was added and incubated overnight. After 24 h the medium was replaced by 100 μL of fresh cell culture medium supplemented with 10 μL of 12 mM MTT (Thermo Fisher Scientific, the Netherlands) solution in phosphate buffered saline (PBS) (Sigma-Aldrich, the Netherlands) and incubated for 4 h. All medium was removed except 25 μL mixed with 100 μL of DMSO (VWR, the Netherlands) and incubated for 10 minutes at 37° C. Absorbance was measured with CLARIOstar microplate reader (BMG Labtech, Germany) at 540 nm. Cell viability was calculated with equation 1 (OD₅₄₀ₑ=measured optical density of extracts of test sample, OD_{540b} =measured optical density of blank).

$$Viability ( \% ) = \frac{100 * {OD}_{540 e}}{O {D}_{540 b}} \qquad ( equation 1 )$$

### Lactate Dehydrogenase (LDH) Cytotoxicity Assay

Cell cytotoxicity was assessed by measuring by the LDH levels in cell culture medium. INS-1E cells were seeded with 5×10⁵ cells/mL in a 96-well (100 μL) and incubated overnight. Next, 10 μL extract sample was added (see MTT assay) and incubated for 45 minutes. 50 μL of each sample was transferred to a 96-well plate and 50 μL reaction mixture (Pierce LDH, Thermo Fisher Scientific, the Netherlands) was added and incubated for 30 minutes at RT while protected against light. 50 μL of stop solution was then added and mixed, followed by measuring absorbance at 490 nm and 680 nm with CLARIOstar microplate reader. Cytotoxicity was calculated with equation 2.

$$Cytotoxicty ( \% ) = \frac{Compound treated LDH activity - Spontaneous LDH activity}{Maximum LDH activity - Spontaneous LDH activity} * 100 \qquad ( equation 2 )$$

### Fourier-Transform Infrared Spectroscopy (FTIR)

Fourier transform infrared spectroscopy (FTIR) (Nicolet iS50-FT-IR, Thermo Scientific, the Netherlands) was used to analyse the chemical composition of the produced membranes. Infrared spectra were recorded at a wavelength range of 400-4000 cm⁻¹. The results were analysed using SpectraGryph (version 1.2.13, Germany) to identify peaks related to chemical bonds.

### Assembly of Encapsulation Devices

Membranes and support rings were cut using a cutting machine (Curio, Silhouette, the Netherlands) into an oval shape (44×28 mm). Devices were produced by placing a support ring between two membranes sheets and annealing them by ultrasonic welding (LPX, Branson Ultrasonics, the Netherlands) inside a custom made mold (IDEE, the Netherlands). The assembled devices were tested for leakage by an air bubble test. Briefly, assembled devices are submerged in water while air (˜4 L/min) is blown through the inlet into the device. Leakages are then detected when escaping air bubbles from the device are observed

### Membrane Diffusion Properties

Membranes were pre-wetted for 2 min in 70% ethanol followed by washing for 2 min with distilled water to remove ethanol. Wetted membranes were placed in In-Line Equilibrium diffusion cells (Sigma-Aldrich, the Netherlands) containing a donor and a receiver chamber. The donor chamber was filled with a PBS solution containing 2 mg/mL 3-5 kDa FITC-dextran (Sigma-Aldrich, the Netherlands), 20 mM D-glucose solution (Sigma-Aldrich, the Netherlands), 0.5 mg/mL FITC-labelled human insulin (Sigma-Aldrich, the Netherlands) or a NaCl solution (150 mM) containing 1 mg/ml IgG solution (Sigma-Aldrich, the Netherlands). The receiver chamber was filled with PBS for the dextran, glucose, or insulin diffusion; or 150 mM NaCl for the IgG diffusion. A 10 μl sample was aliquoted from both chamber at 0, 5, 10, 20, 30, 60, 120, 180 minutes, in the case of insulin diffusion an additional sample at 1440 minutes was taken. Quantitative analysis of diffused FITC-dextran and FITC-insulin was determined based on fluorescent intensity using CLARIOstar microplate reader (excitation and emission wavelength=480 nm and 530 nm). The amount of glucose was measured using colorimetric glucose assay (Bio-connect Diagnostics, the Netherlands) according to manufacturer's protocol. Briefly, samples were collected and diluted in PBS. 50 μL of glucose solution was incubated with 50 μL of reaction mix for 20 minutes at 37° C. Absorbance was measured using CLARIOstar microplate reader (excitation wave length=555 nm). The amount of IgG was measured with an IgG ELISA (Thermo

Fisher Scientific, the Netherlands) according to manufacturer's protocol. The flux values were calculated with equation 3 to get insight into how much of the molecule of interest was diffused over the membrane at a specific time (C_{Receiver}=concentration at receiver chamber, V_{Reeiver}=volume of receiver chamber, A=area of membrane, t=time). The diffusion coefficient was calculated with equation 4 (ΔC=concentration difference between donor and receiver, I=thickness of membrane). Each condition was tested at least four times.

$$flux ( {gm}^{- 2} {s}^{- 1} ) = \frac{{C}_{Receiver} ( g {m}^{- 3} ) * \frac{{V}_{Receiver} ( {m}^{3} )}{A ( {m}^{2} )}}{t ( s )} \qquad ( equation 3 )$$

$$diffusion coeffient = \frac{flux ( g {m}^{- 2} {s}^{- 1} )}{Δ C ( {gm}^{- 2} )} * l ( m ) \qquad ( equation 4 )$$

### Oxygen Diffusion Properties Inside the Device

Before the devices were loaded with medium, the assembled devices were pre-wetted for 2 min in 70% ethanol, followed by 2 min washing with distilled water to remove ethanol. Oxygen diffusion was measured by loading the device with 500 μL of RPMI cell culture medium. Medium loaded devices were heat-sealed and placed inside a 100 ml beaker containing 100 mL of fresh cell culture medium. The beaker containing the device was then placed at 37° C. in a humidified incubator with 5% O₂. The oxygen concentration within the devices and the outside were recorded during 24 hours, using a previously calibrated needle-type oxygen micro-sensor (NTH-PSt7, Presens, Germany) connected to an oxygen meter (OXY-10, Presens, Germany). Devices were kept submerged in media during the entire oxygen measurement duration. Each condition, control and both devices, were tested three times independently.

### Immunoprotective Capacity of Membranes

Membranes were mounted onto 0.33 cm² empty transwell inserts (Corning, the Netherlands) with glue (Bison, the Netherlands) and O-rings (Eriks, the Netherlands). After allowing the glue to dry overnight, inserts were sterilized in 70% ethanol. Human primary macrophages were seeded (100,000 cells in 100 μL) onto the top compartment of each inserts in a 24-well plate, 600 μL medium was added to each well. Cells were cultured for 24 h (37° C., 5% CO₂). Cells which migrated to the bottom compartment were counted manually using Neubauer hemocytometer. To observe cell adherence and infiltration through the membranes, membranes were fixed with 3.6% formaldehyde (VWR, the Netherlands) in PBS for 30 minutes at RT, followed by multiple rinses in fresh PBS, membranes were then cut from the inserts. Samples were dehydrated with increasing ethanol concentrations (30, 50, 70, 90, 96, 100 and 100% ethanol) (VWR, the Netherlands) for 10 minutes each. Dehydrated samples were processed using an automated critical point dryer (Leica, the Netherlands). Subsequently, the dried membrane samples were prepared and imaged using scanning electron microscopy as described previously.

Islet Seeding into the Encapsulation Devices

Devices were sterilized by overnight incubation in 70% ethanol, followed by washing with sterile PBS. Before seeding islets into the devices, devices were incubated in medium overnight (37° C., 5% CO₂). 3,000 human islet equivalent (IEQ) in 500 μL medium was seeded inside each device. For the control samples, 30 IEQ were seeded in 12 μm cell culture inserts (Merck, the Netherlands). The seeding inlet was closed with heat sealing (Durapak, United States). Devices were cultured for 8 days (37° C., 5% CO₂) and medium was refreshed every other day.

### Human Islet Viability

The viability of human islets was assessed using live-dead fluorescent dyes, calcein AM and ethidium homodimer-1 (Thermo Fisher Scientific, the Netherlands) for live and dead cells, respectively. After 8 days of culturing, devices were cut-opened and islets were flushed out of the device with PBS. The islets were then incubated with 2.5 μM calcein AM and 4 μM ethidium homodimer-1 in PBS for 30 minutes (while protected against light) and visualized under fluorescent microscopy (Eclipse Ti—S inverted microscope, Nikon, the Netherlands). The percentage of viability was quantified by dividing the area of live cells by the total area of cells (live and dead).

### Human Islet Function During Glucose Stimulation

To assess the insulin release of human islets inside the devices a glucose stimulated insulin secretion (GSIS) assay was performed. Krebs buffer was prepared by dissolving 25 mM HEPES (Sigma-Aldrich, the Netherlands), 115 mM Sodium Chloride (Sigma-Aldrich, the Netherlands), 26 mM Sodium Bicarbonate (Sigma-Aldrich, the Netherlands), 5 mM Potassium Chloride (Sigma-Aldrich, the Netherlands), 1 mM Magnesium Chloride Dihydrate (Sigma-Aldrich, the Netherlands), 25 mM Calcium Chloride Dihydrate (Sigma-Aldrich, the Netherlands) with 2% Bovine Serum Albumin (BSA) (VWR, the Netherlands) in MilliQ at pH 7.3-7.5 and filtered sterile. Low and high glucose solutions contained 1.67 and 16.7 mM D-glucose in Krebs buffer respectively. After 8 days of culturing, devices and islets were washed three times in low glucose solution, followed by 1 h pre-incubation in low glucose (37° C., 5% CO₂). Next, devices and islets were exposed to low-high-low glucose solutions for 1 h each (37° C., 5% CO₂) after which supernatant was collected and stored at −30° C. until analysis was performed. Devices and islets were washed three times with low glucose solution after high glucose exposure. After the final incubation step devices were opened and islets were exposed to acid ethanol to release all insulin inside the cells. Insulin secretion was determined with human insulin ELISA kit (Mercodia, Sweden, or Sanbio, the Netherlands) according to manufacturer's protocol. The glucose stimulation index was calculated by dividing the insulin secretion in high glucose solution by the basal insulin secretion in low glucose solution.

### Data Analysis

Results are presented as mean±standard deviation (STD), unless otherwise specified. Experiments were performed in triplicate unless specified. For each experiment three samples were taken per experimental condition. Statistical analysis was performed by using GraphPad Prism 9 (GraphPad Software, United States). Statistical difference between two groups was assessed using an unpaired student t-test; and among multiple groups using a Kruskal-Wallis, or a one-way ANOVA test with multiple comparison. Statistical significance was considered when p<0.05.

### Results

### Fabrication and Physiochemical Characterization of Membranes

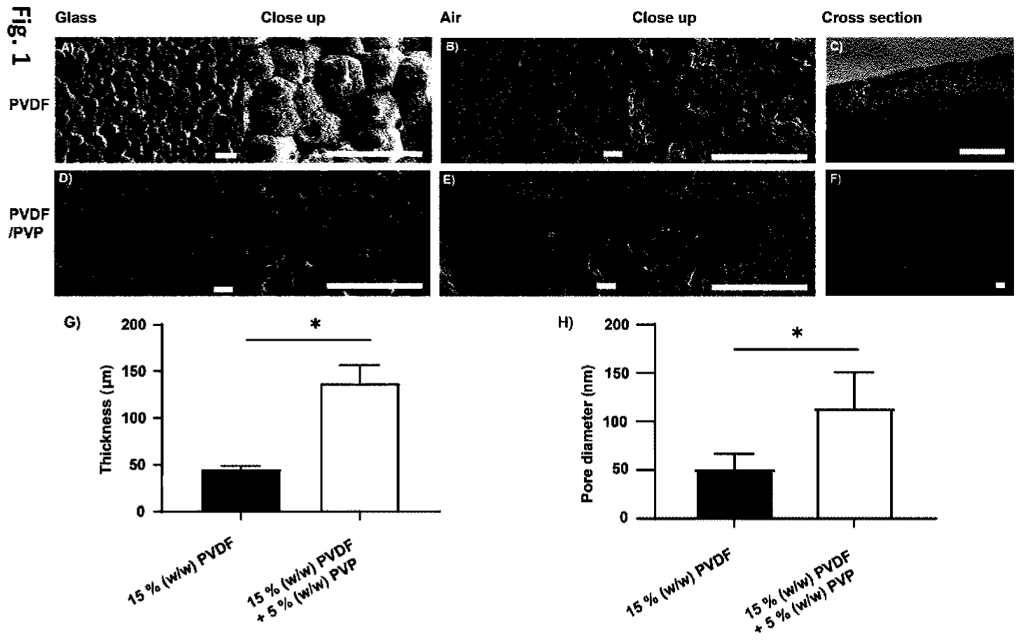

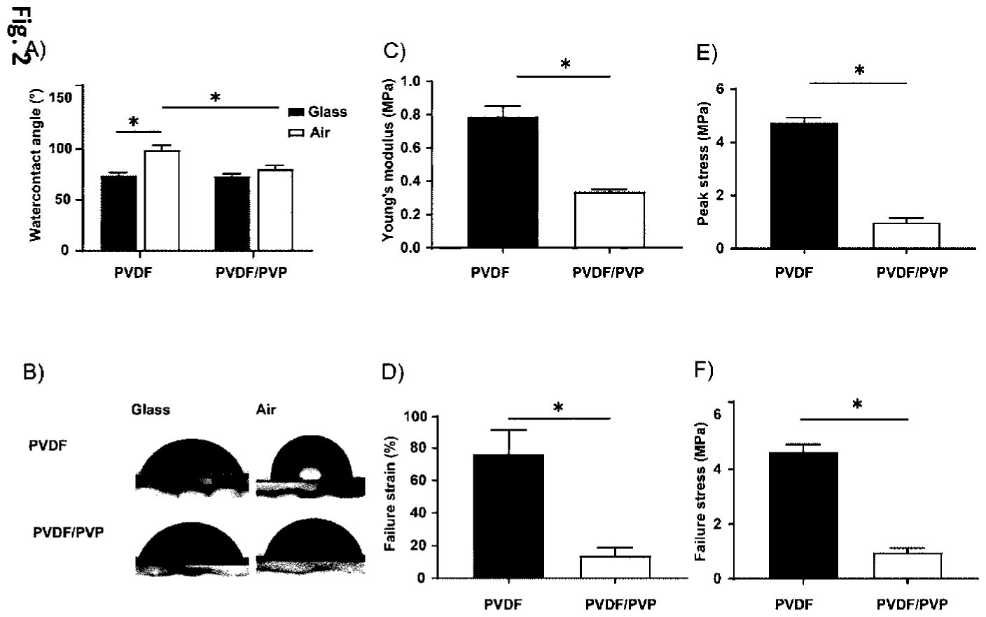

Thin and porous membranes were made using a solvent/non-solvent casting method, by casting 15% (w/w) PVDF+5% (w/w) PVP (PVDF/PVP) and 15% (w/w) PVDF (PVDF alone) solutions onto glass plates. The surfaces of both PVDF membranes, with and without PVP, have a smooth and uniform appearance. Larger pores were observed on the glass side (FIGS. 1A and D) compared to the air side (FIGS. 1B and E) of the membranes. The glass-side pores in PVDF/PVP membranes had an average diameter of 0.51±0.15 μm, while the glass-side pores in PVDF membranes had an average diameter of 0.25±0.12 μm (data not shown). The presence of PVP also increased pore size on the air-exposed side (FIGS. 1B and E), with a mean diameter of 113±4 nm compared to PVDF-alone membranes (51±2 nm), a more than two-fold increase (FIG. 1H). The cross-section of PVDF-alone membranes was denser, and thinner (45 ±4 μm), compared to PVDF/PVP membranes (137±19 μm, FIG. 1C, F, G).

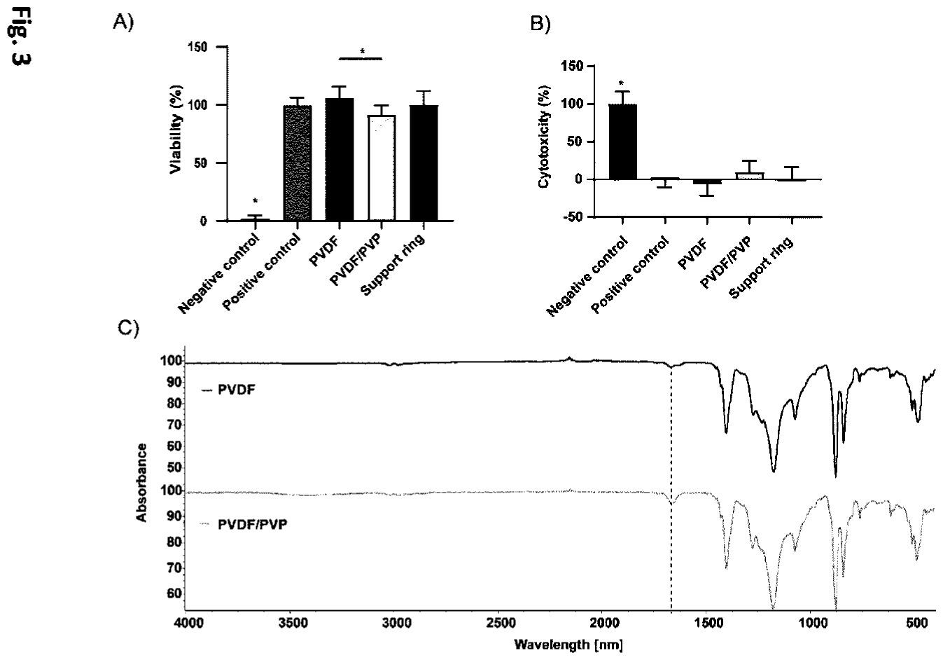

Contact angle measurements were performed by static sessile drop method to determine the hydrophilic/hydrophobic properties of produced membranes. Unlike that of the glass-exposed side (both approximately xx), the surface wettability of the air-exposed side of the PVDF/PVP membranes was significantly lower compared to PVDF membranes (80±3° v 99±4°; FIGS. 2A and B).

The mechanical properties of the produced membranes were determined with mechanical tensile testing. PVDF/PVP membranes showed significantly less mechanical strength compared to PVDF membranes, as determined by the Young's modulus ((0.32±0.04 MPa for PVDF/PVP and 0.87±0.11 MPa for PVDF alone; FIG. 2C) and peak stress measurements (0.97±0.03 MPa for PVDF/PVP and 4.95±0.16 MPa for PVDF alone; FIG. 2D). Moreover, both failure strain (FIG. 2E) and failure stress (FIG. 2F) were significantly lower in PVDF/PVP membranes compared to PVDF-alone membranes: 0.92±0.06 MPa v. 4.78±0.07 MPa and 11±1% v. 84±8%, respectively.

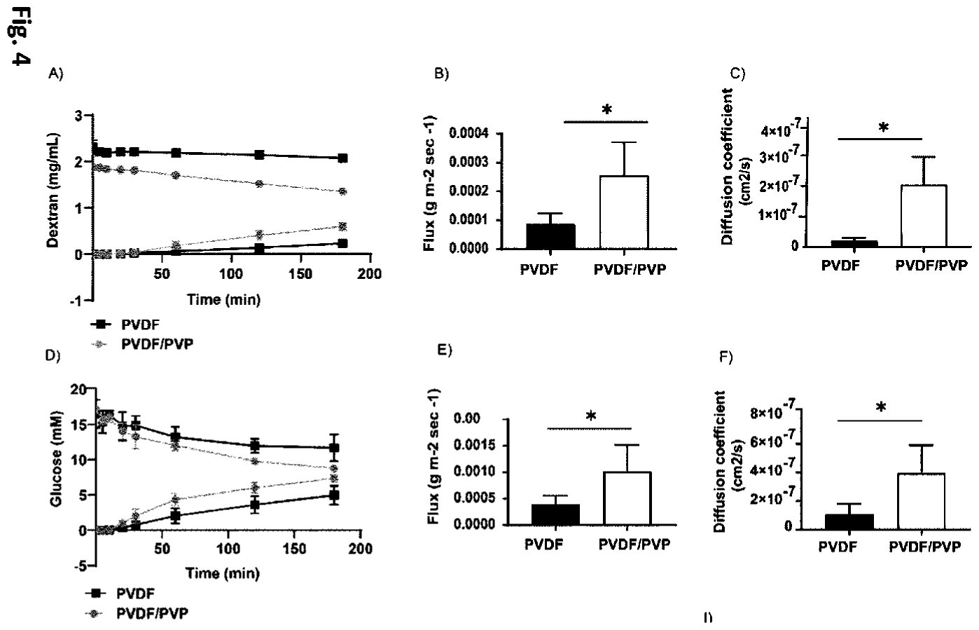

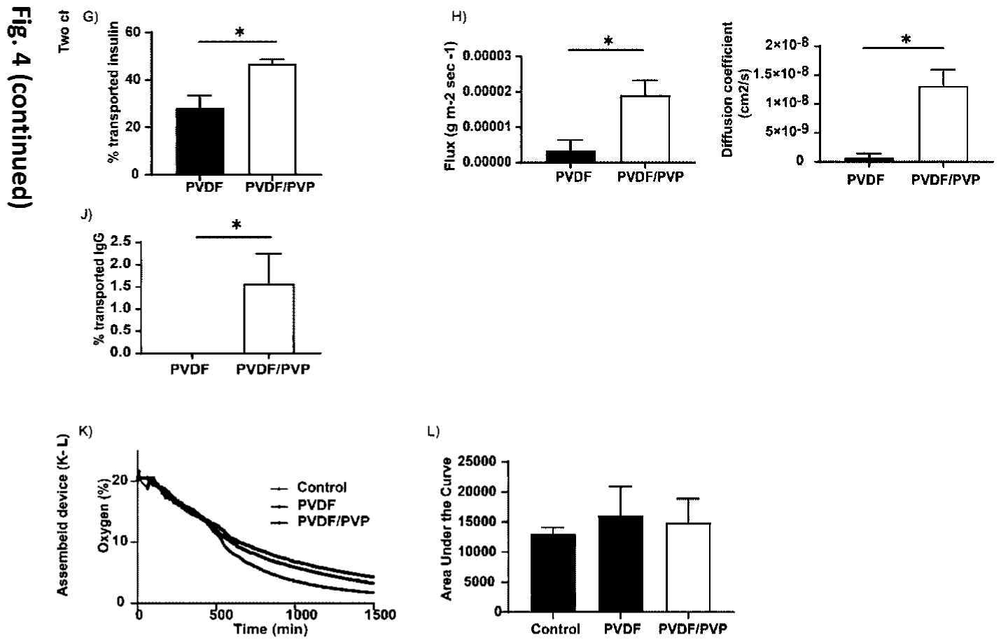

Cytotoxicity of membranes To determine the cytotoxicity of produced PVDF membranes and support rings on INS-1E cells, LDH and MTT assays were performed. Cell viability was slightly but significantly lower in PVDF/PVP membranes compared to PVDF-alone membranes (92±8 vs 106±9%; FIG. 4A). Neither membrane showed significant differences in cytotoxicity compared to the positive control (−7±15 vs 10±14% for PVDF and PVDF/PVP membranes, respectively; FIG. 4B). Although PVP is water soluble, residual and potentially cytotoxic PVP particles may have remained after membrane fabrication that influenced our cytotoxicity measurements. Therefore, FTIR analysis was performed to determine if there were still PVP residues present (FIG. 3C). In PVDF-alone membranes, peaks were present at ˜1400 cm⁻¹ and ˜1180 cm⁻¹, corresponding to CH2 and CF2 stretching groups present in the base structure of PVDF. In PVDF/PVP, an additional peak at ˜1660 cm 1 was observed, corresponding to the C═O group present in PVP (green dotted line in FIG. 3C).

### Diffusion of Small Molecules Through Membranes

To gain insight into the transport of different molecules across membranes, we tested four molecules: dextran, glucose, insulin, and IgG. The membranes were placed in-between two chambers, a donor and a receiver chamber. The concentration measurements of the different molecules were taken from each chamber at predefined time points to calculate the diffusion kinetics. The diffusion of dextran (3-5 kDa) through PVDF/PVP was nearly three-fold higher than in PVDF membranes (FIG. 4A), and the calculated flux and diffusion coefficient were significantly higher (FIGS. 4B and C). Similarly, glucose molecules diffused faster through PVDF/PVP membranes, reaching equilibrium (equal in the two chambers) after 3 h, in contrast to only 30% of the glucose diffusing through PVDF membranes (FIG. 4D) at the same time point. This observation was confirmed by the significantly higher glucose flux of 10*10⁻⁴ g m⁻² sec⁻¹ and diffusion coefficient of 4.0*10⁻⁷ cm² s⁻¹ for the PVDF/PVP membrane compared to PVDF alone (4*10⁻⁴ g m⁻² sec⁻¹ and 1.1*10⁻⁷ cm² s⁻¹, respectively; FIGS. 4E and F). Glucose was not absorbed by the membrane, as the total concentration of glucose remained the same over time. FITC-labelled insulin reached equilibrium after 24 h in PVDF/PVP membranes as 47±2% of insulin was diffused, compared to whereas 28±5% was diffused in PVDF membranes at the same time point (FIG. 4G). The insulin diffusion capacity was confirmed by significantly higher flux of 1.91*10⁻⁵ g m 2 sec⁻¹ and diffusion coefficient of 1.31*10-8 cm² s⁻¹ in PVDF/PVP membranes compared to PVDF membranes (0.35*10⁻⁵ g m 2 sec-1 and 0.08*10-8 cm² s⁻¹, respectively; FIGS. 4H and I). For IgG, only 1.6±0.7%, permeated through the PVDF/PVP membrane, but could not be detected in the receiver chamber of the PVDF-alone membrane, suggesting no diffusion (FIG. 4J).

The transport of oxygen was not significantll different in free solution (cell culture medium) compared to both PVDF and PVDF/PVP based membrane devices, as areas under the curve were similar (FIGS. 4K and 4L).

### Immunoprotective Function of PVDF Membranes

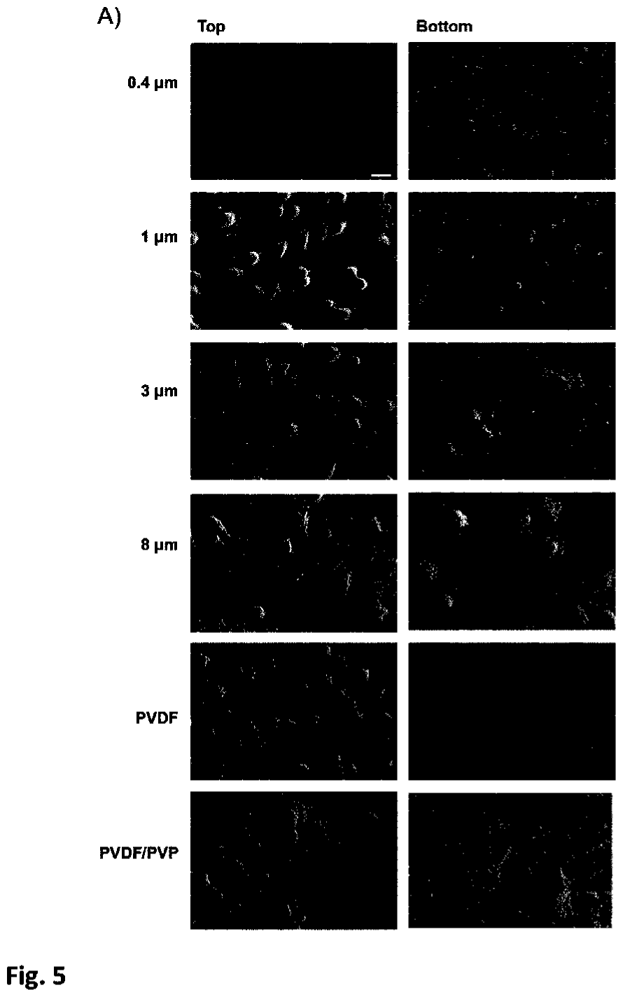

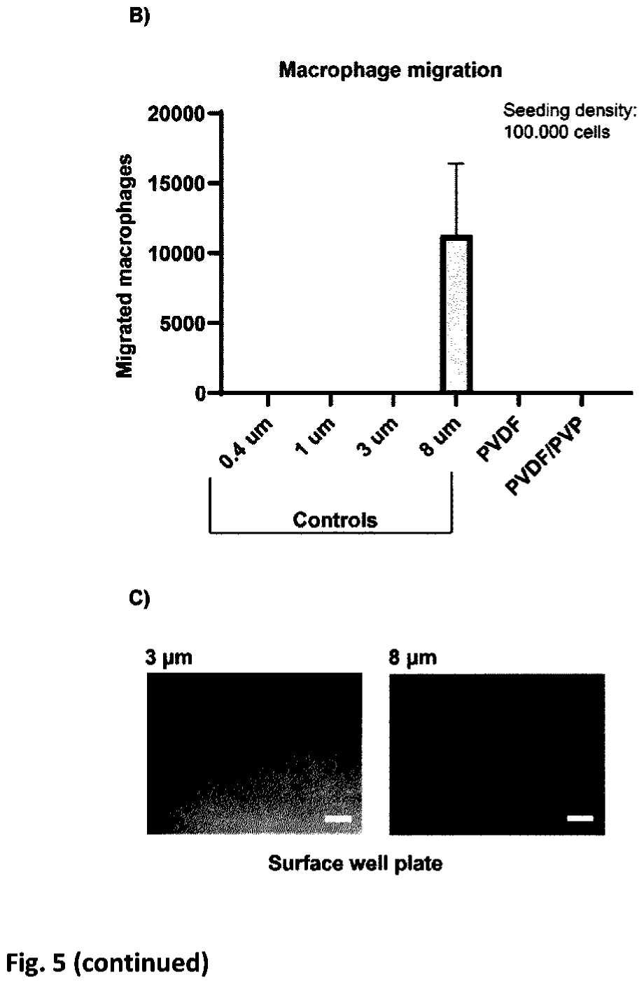

To evaluate the immunoprotective properties of the produced membranes (PVDF and PVDF/PVP), we assessed the migration of macrophages from top to bottom of transwell inserts fixed with a specific membrane. As shown with SEM images (FIG. 5A) no macrophages were detected on the bottom side PVDF and PVDF/PVP membranes, as well as those with 0.4 μm pores. Macrophages were detected on the bottom side of membrane with 3 and 8 μm pores, though only with the 8 μm pores macrophages were able to fully migrate to the bottom well (FIGS. 5B and C). The nuclei of the human macrophages were not able to migrate through 1 μm pores; however, the pores were large enough to enable the migration of filopodia, the cells antennae that sense the extracellular environment (FIG. 5A).

### Assembly of the Immunoprotective Encapsulation Devices

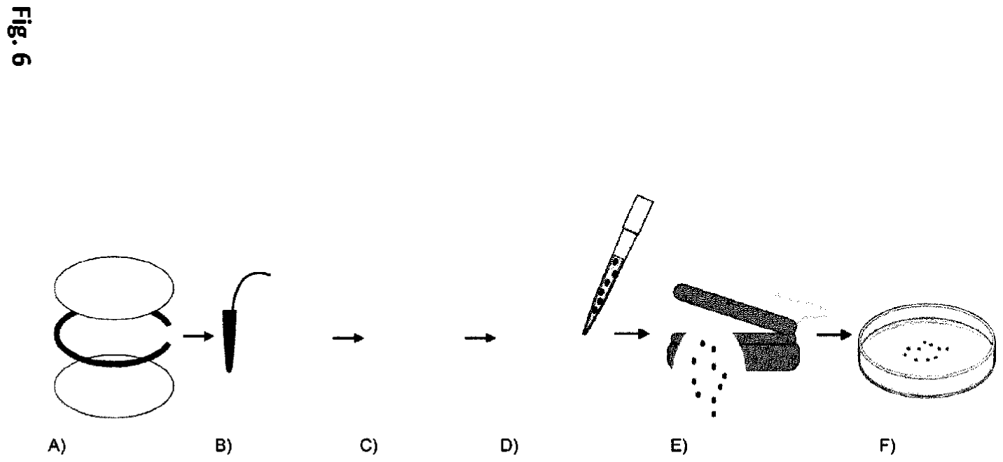

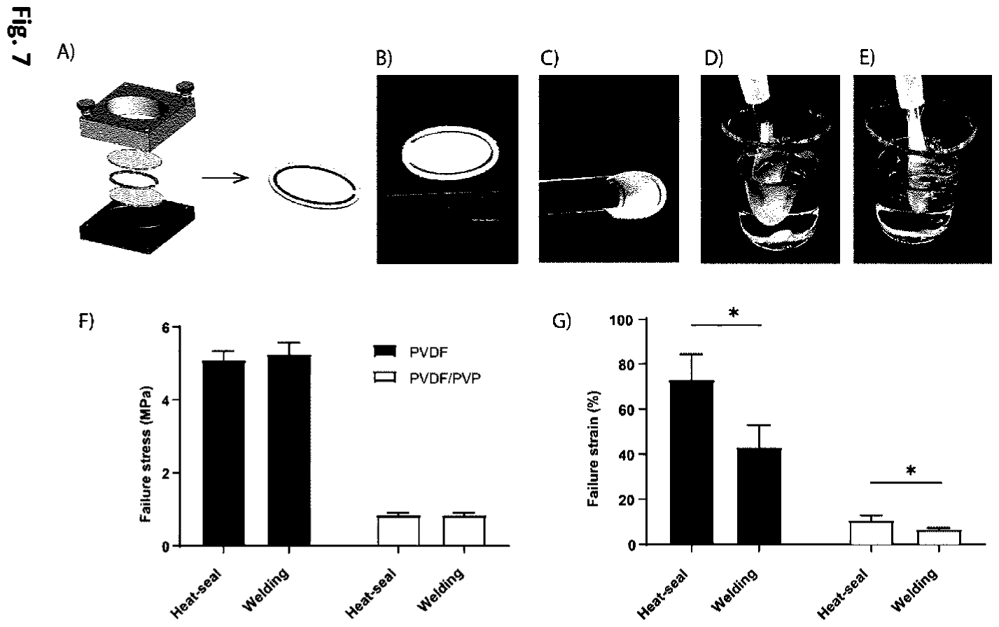

To assemble the encapsulation devices, two membrane sheets were welded together with high-frequency ultrasonic welding or heat sealing using a support ring in-between as illustrated in FIG. 6A. A small part was not welded to serve as an inlet for loading cells into the device (FIG. 6D). Each device was tested for leakage with a pressurized air bubble test. Welding defects were detected by visually inspecting the escape of air bubbles (Supplementary FIG. 4D). Devices with welding defects were not used for further experiments.

### Viability and Function of Human Islets Inside the Encapsulation Devices

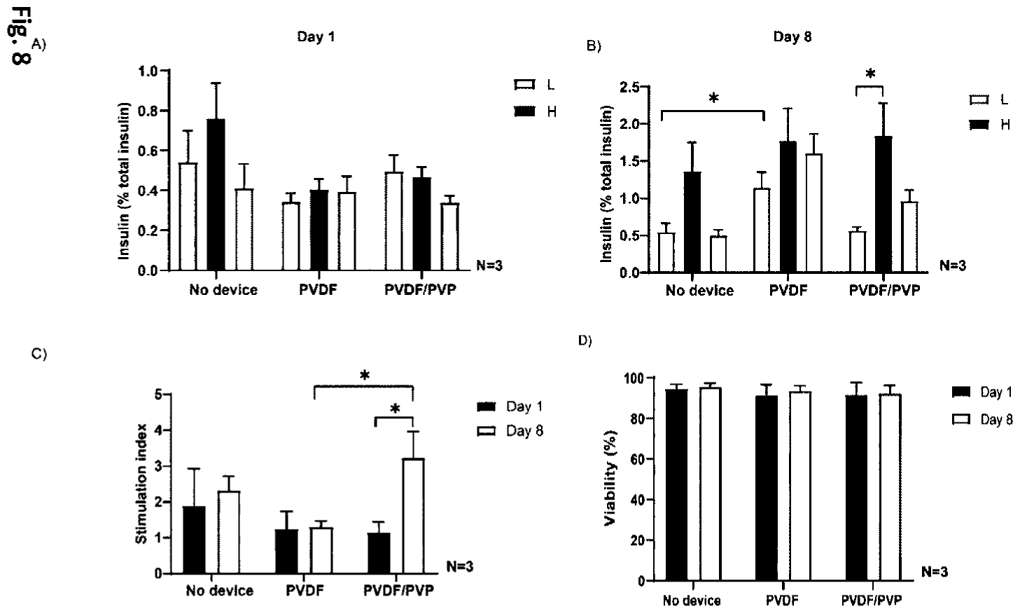

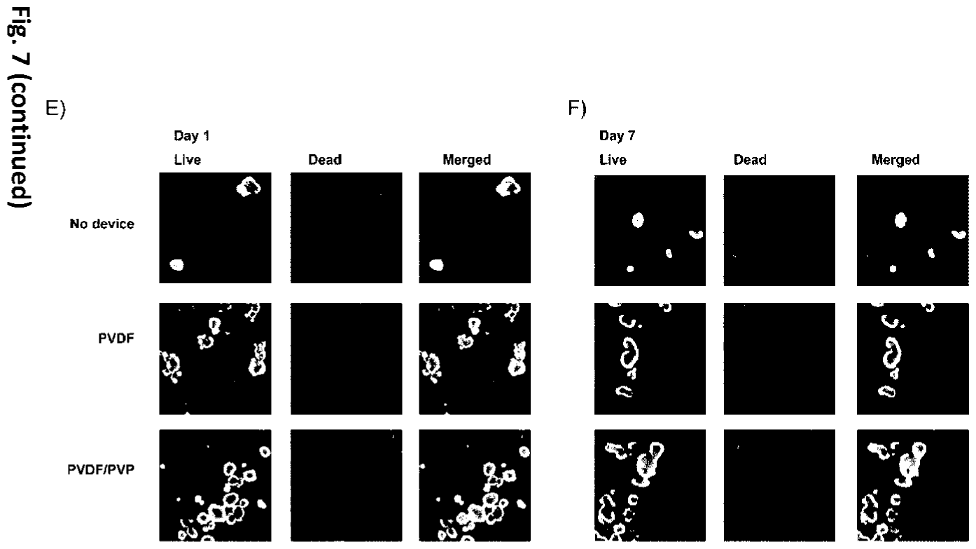

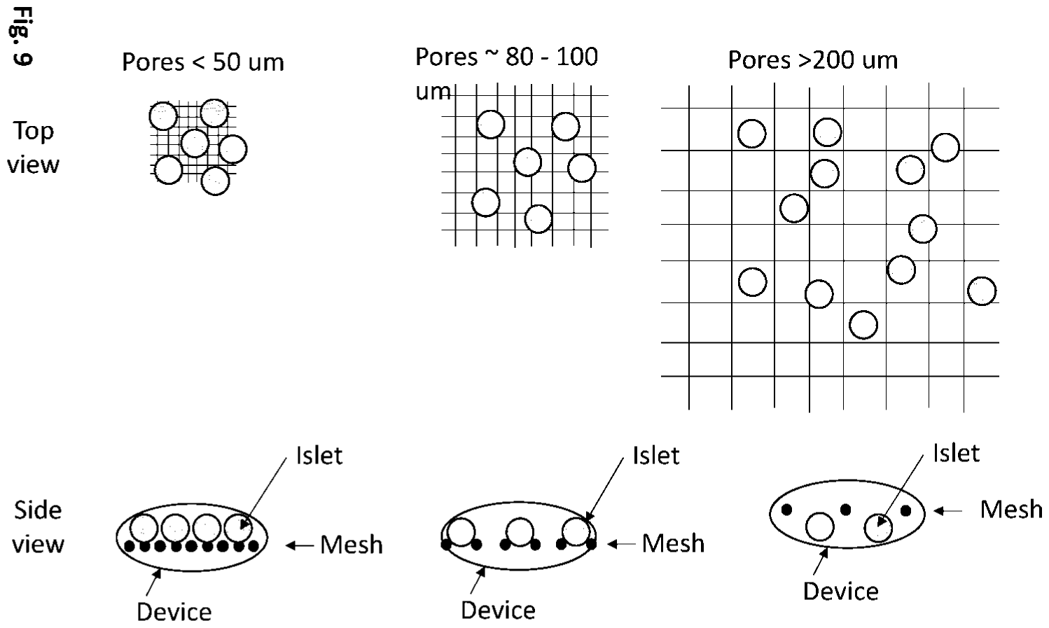

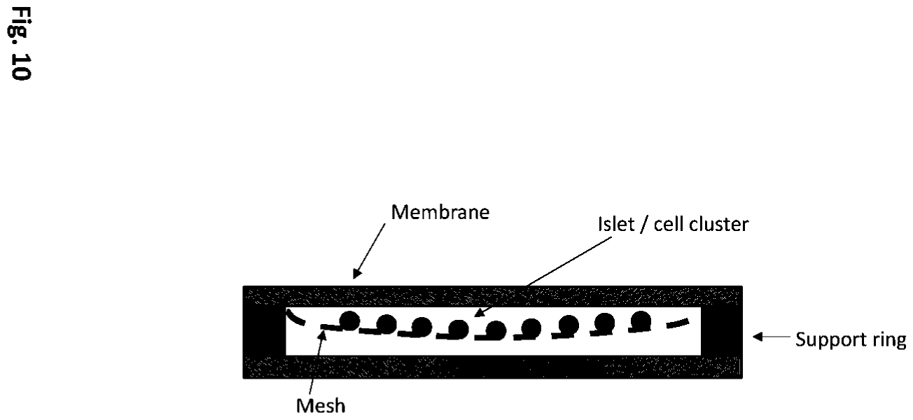

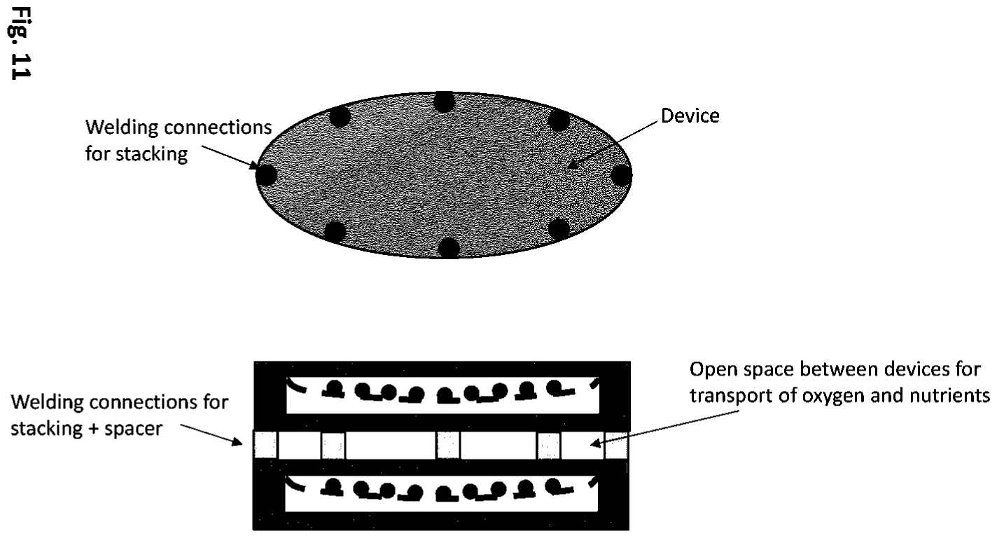

Human islets were cultured for 8 days inside the closed immunoprotective devices to assess their viability and islet function with live-dead staining and GSIS assays respectively. No significant difference in viability was observed between islets cultured inside the PVDF and PVDF/PVP devices, compared to controls (FIGS. 8A and B). 92±4% of the human islets were viable after 8 days of culturing inside the PVDF/PVP encapsulation device. In the control condition, the islets responded to changing glucose levels by the increased insulin release in high glucose condition after 8 days with a stimulation index of 2.3±0.4 (FIGS. 8C and D). An increased insulin secretion during high glucose condition was observed in PVDF/PVP devices (FIG. 8C). The stimulation index of PVDF/PVP devices was significantly higher than the stimulation index of PVDF devices, 3.2±0.7 vs 1.3±0.2 respectively (FIG. 8D).

### Example 2

### Using a Mesh (80 μm) for Better Cell Distribution

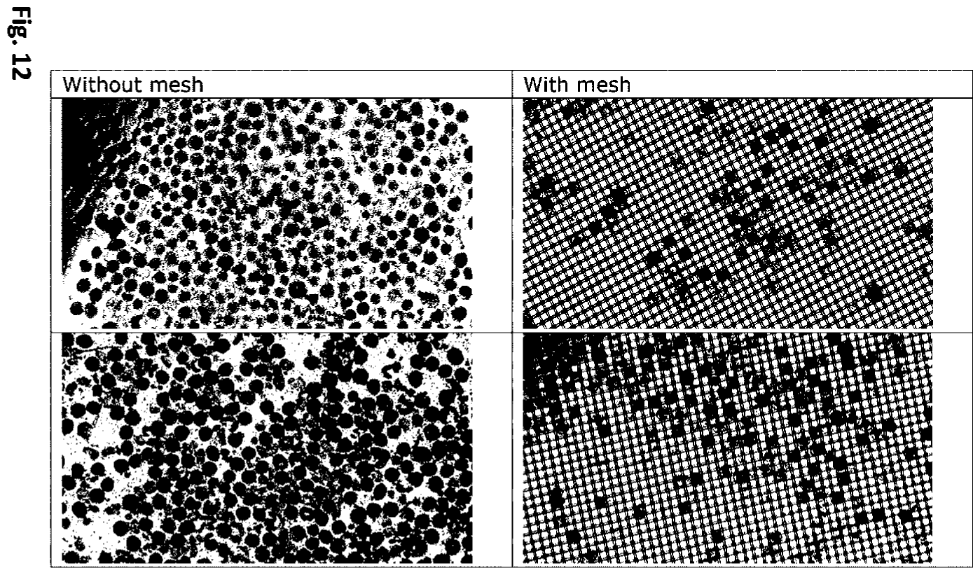

Pancreatic beta cell clusters were grown in the presence or absence of a mesh. In the condition without the mesh, it can be observed that all the cell aggregates are located close to each other, which will result in further cell aggregates when cultured for longer periods. In the condition with the mesh, it can be observed that the cell aggregates are more spread around. This will prevent that the cell aggregates can cluster together when cultured for longer periods. Concluding that the mesh enhance the cell distribution in order to prevent clustering. Results are depicted in FIG. 12. Images were made at the same magnification and the same concentration of aggregates were seeded onto the same culture area.

### Test in Encapsulation Device

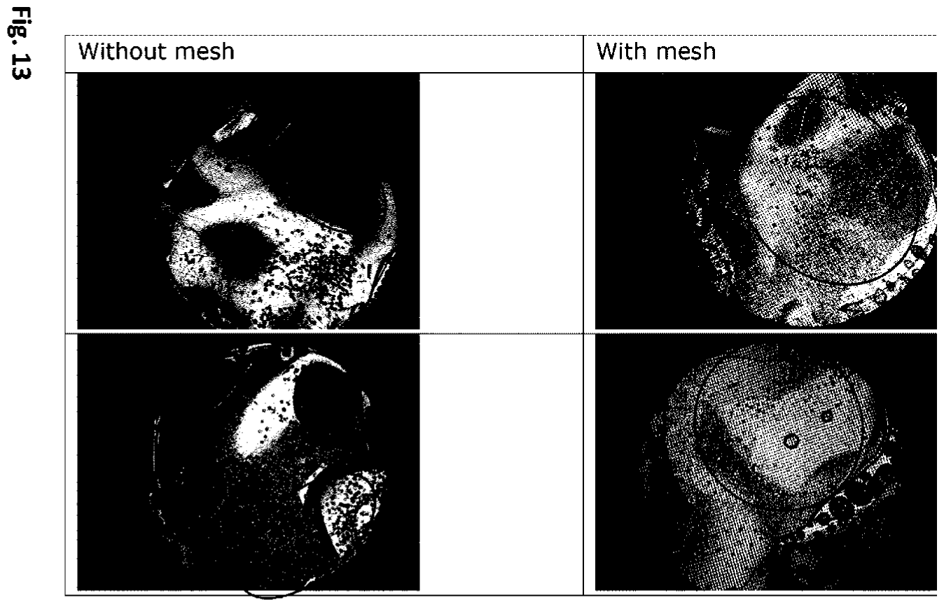

The effect of the mesh when placed inside the closed encapsulation devices has a similar outcome compared to the Petri dish experiment (see above). In the devices without the mesh the cell aggregates are located close to each other (see circle). In the devices with the mesh the cell clusters are more evenly distributed inside the device and cluster less together. Concluding that the use of a mesh can be beneficial for a better cell aggregate distribution inside the devices. Results are depicted in FIG. 13. Images were made at the same magnification and the same concentration of aggregates were seeded onto the same culture area.

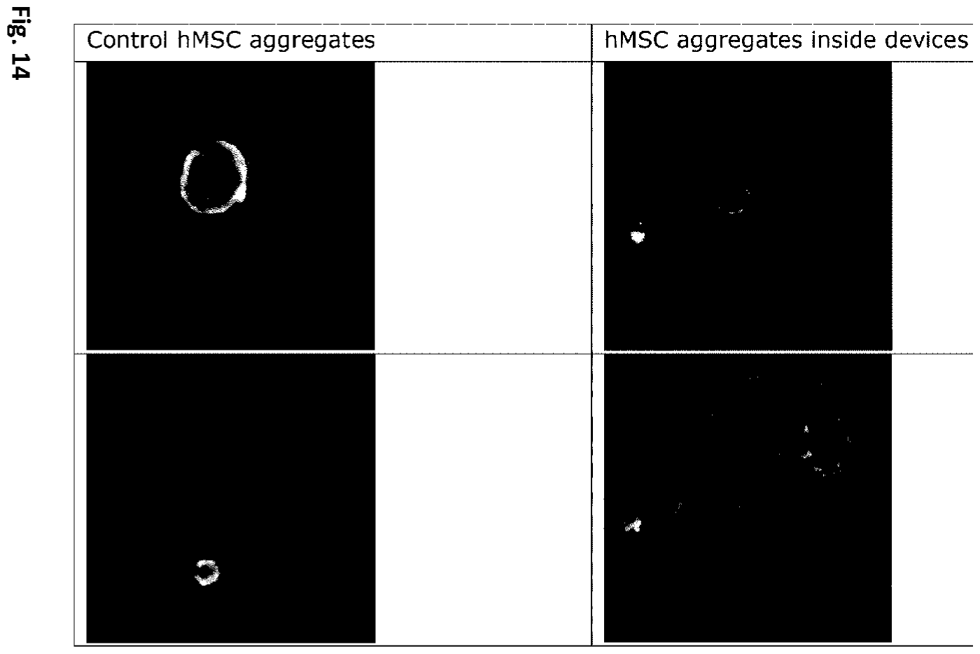

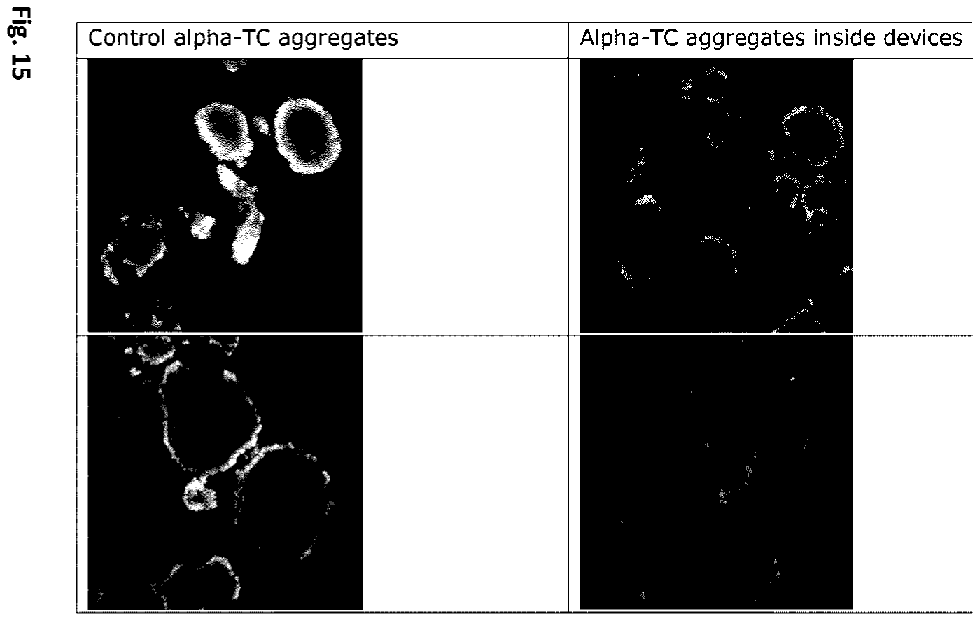

Cell cluster viability after 7 days of culturing inside the closed encapsulation device Both of the clustered cells (Alpha-TC cells and hMSC) remained viable after 7 days of culturing inside the closed encapsulation devices. Viability levels were similar to cell aggregates not cultured inside the encapsulation devices. It can be concluded that the encapsulation device can serve to not only keep islets of Langerhans alive, but also different cell types can be cultured inside the closed encapsulation device without influencing the cell cluster viability. Results are depicted in FIGS. 14 and 15.

### Example 3-Different Mesh Sizes

### Reference Experiment 1

Goal: observe the islet distribution inside the devices

### Method:

Similarly sized devices were made with and without a mesh. The used mesh had a pore diameter of 400 μm. The mesh was placed between 1 membrane sheet and the support structure. Approximately 3.000 islet equivalence (IEQ) were seeded in 500 μL into the device. For devices containing the mesh: islet cells were seeded on top of the mesh (so that the islets could get trapped inside the mesh)

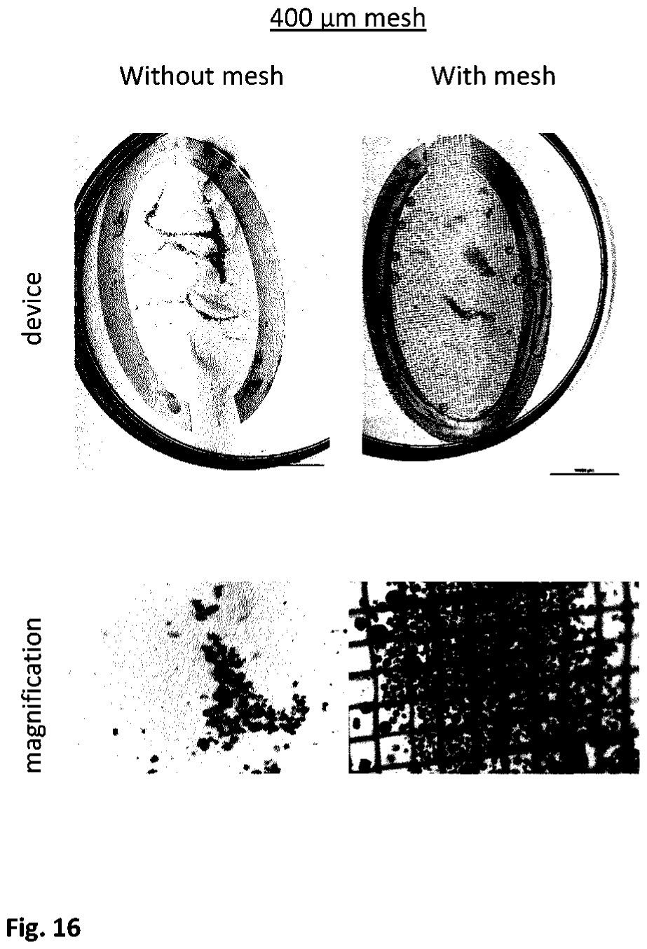

Observation: the device and cells are depicted in FIG. 16.

Conclusion: The presence of this mesh inside the devices did not help the distribution of the islets. This is because the mesh structure is very open and the islets fall through the openings and not holding the islets, as depicted in the magnified image.

### Reference Experiment 2

Goal: Produce transparent devices with a ˜50 μm mesh inside. To test the aim is the mesh helps to prevent clumping of the pseudo-islets seeded in devices and whether the pores are small enough to prevent that the pseudo-islets fall through the pores.

Method: 2 devices were made with a ˜50 μm mesh. The mesh was placed between 1 membrane sheet and the support structure. Approximately 2.500 pseudo islets were seeded in 500 μL into the device. For devices containing the mesh: islet cells were seeded on top of the mesh (so that the islets could get trapped inside the mesh)

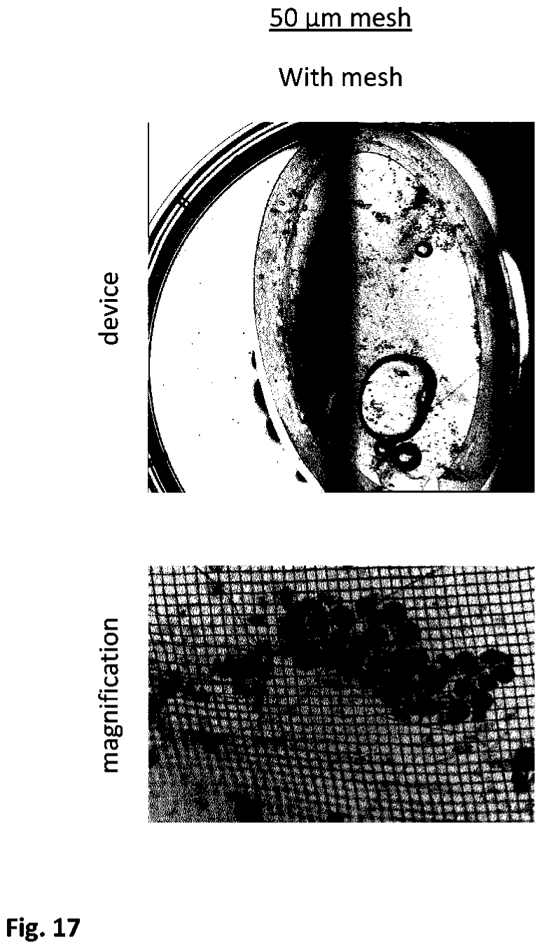

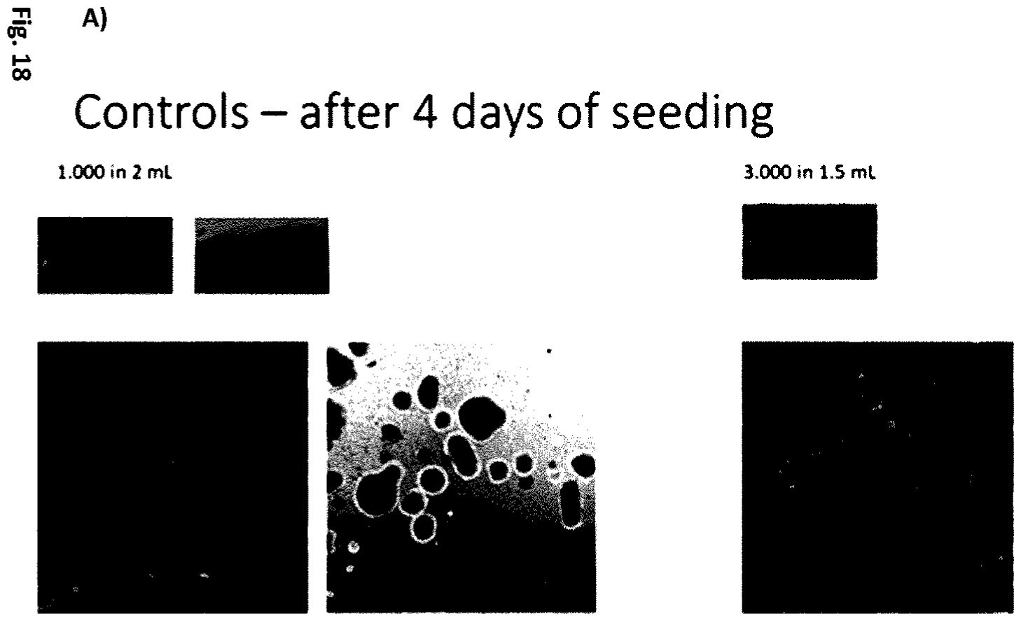

Observation: the device and cells are depicted in FIG. 17. The seeded pseudo-islets did not fall through the present pores. The pseudo-islets were not homogenously distributed over the surface.

Conclusion: The seeded pseudo-islets did not fell through the mesh; this was possible because the pore diameter was smaller than the diameter of the seeded pseudo-islets. The distribution of the pseudo-islets was better in devices with a mesh compared to the device without a mesh. However, the pseudo-islets had the tendency to settle close to each other and, were not captured inside the present pores. Therefore, the seeded cells only partly remained separated from each other. For better distribution, slightly larger pores are required.

Experiment 3 Goal: Evaluate pseudo-islet distribution using a 80 μm mesh in a well plate and compare to free-floating control.

### Part 1:

Method: The meshes (80 μm pores) were placed in a 6 well plate. Seed pseudo-islets on top of pseudo-islets on top of a mesh with 80 μm pores. Pseudo-islets have a diameter of approximately 150 μm. Note: non tissue treated 6 well plates were used as control.
Seeding densities:
   C1: 1.000 aggregates in 2 mL.
   C2: 3.000 aggregates in 2 mL.
   M1: 1.000 aggregates in 1 mL.
   M2: 1.000 aggregates in 2 mL.
   M3: 1.000 aggregates in 5 mL.
   M4: 3.000 aggregates in 1.5 mL

Results: the aggregates in the control have all the freedom to move and try to center in the middle. When the mesh is present, some of the aggregates are trapped inside the mesh structure and some of the aggregates are still floating around. It seems that the higher the volume the more aggregates are floating resulting that they are being centered in some areas on the mesh.

Part 2: After 4 days of culturing a live/dead staining was performed to visualize pseudo-islet viability.

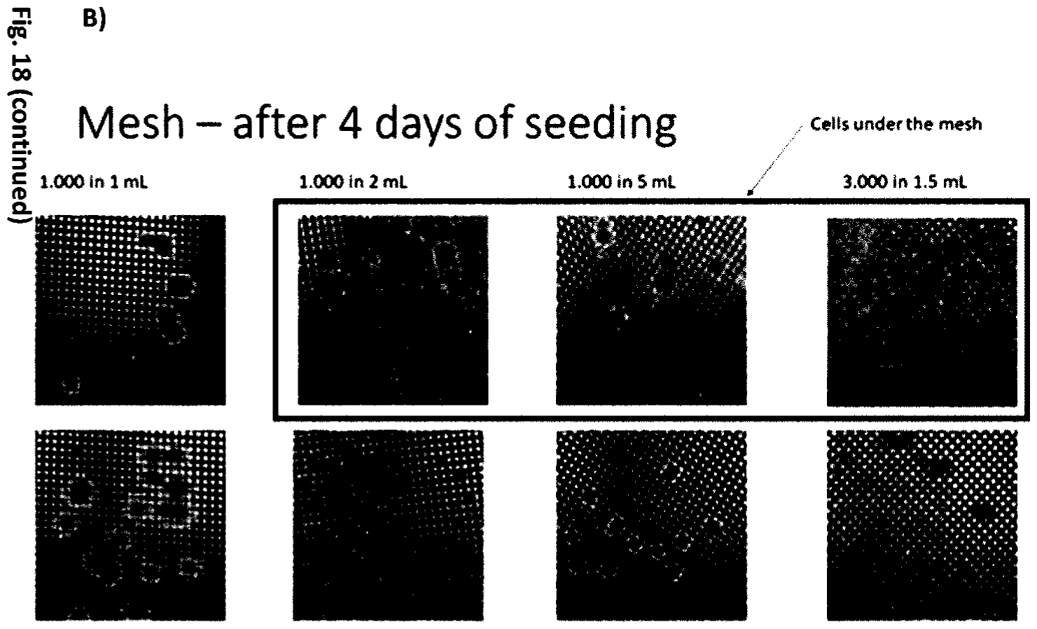

Observation & images—the results are depicted in FIG. 18. C1: some bigger clusters were visible, those were floating. Some of the clusters did adhere to the well plate. C2: most of the clusters were floating and not adhered to the well plate. The viability was a little lower. The clusters were less round as the C1. M1: Most of the clusters did adhere to the mesh and spread out. Only a few aggregates went under the mesh. M2: some of the clusters went under the mesh and started to aggregate. However, most of them were still on top and attached and separated by the mesh. M3: a relative large amount of clusters went under the mesh and started to aggregate, although no big clusters were formed. The cluster on top of the mesh remain separated. M4: a relative large amount of clusters went under the mesh and started to aggregate, although no big clusters were formed. The cluster on top of the mesh remain separated. Conclusion: The pseudo-islets in the control condition merged together over a time period off 4 days, resulting in the formation of large clusters (C1 & C2). The pseudo-islets in the mesh condition remained in place and did not form larger structures. Viability remained high, so the mesh did not have an influence on the viability of the encapsulated pseudo-islets. Therefore, the mesh demonstrated positive effect in keeping pseudo-islets separated.

From the experiments in this example can be concluded that the mesh size is preferably less than 400 μm, for example 350, 300, 250, 200 or 150 or less μm. Further it can be concluded that the mesh size is preferably above 50 μm, for example 60, 70, 75 or 80 μm or above.

1. An implantable device of the immunoprotective type for implanting cells or cell aggregates in a subject, wherein the device comprises a bottom membrane and a top membrane, wherein the bottom membrane and the top membrane are joined together with a support structure such that the bottom membrane, top membrane and the support structure form an enclosed inner space not accessible by the immune system when implanted, and
wherein the bottom membrane and the top membrane comprise a plurality of pores extending through the membrane to allow diffusion of nutrients and oxygen to the inner space, and
wherein the device is characterized by the presence of a mesh positioned in the inner space of the device such as to provide a support for cells or cell aggregates when the cells or cell aggregates are comprised in the inner space of the device, such that the cells do not clump, wherein the mesh has a pore dimension between 60 and 300 μm.

2. The implantable device according to claim 1, wherein the bottom membrane and/or the top membrane and optionally the support structure are made from a non-water soluble polymer, preferable of polyvinylidene fluoride (PVDF), polyethersulfone (PES), poly(ethylene oxide terephthalate)/poly(butylene terephthalate) (PEOT/PBT) or PVDF mixed with a water soluble co-polymer, preferably wherein the membranes further comprise a pore forming reagent, more preferably wherein the pore forming reagent is polyvinylpyrrolidone (PVP).

3. The implantable device according to claim 1, wherein the pore size of the bottom membrane and the top membrane is between 10 and 500 nm, preferably between 50 and 450 nm more preferably between 100 and 400 nm.

4. The implantable device according to claim 1, wherein the bottom membrane and the top membrane have a thickness of between 10 and 200 nm, preferably between 35 and 200 nm, more preferably between 40 and 160 nm.

5. The implantable device according to claim 1, wherein the mesh is constructed from a non-degradable polymer, preferably wherein the non-degradable polymer is selected from nylon, PVDF, PTFE, PP, PE, or PET.

6. The implantable device according to claim 1, wherein the mesh has a pore dimension of between 60 and 200 μm, preferably between 70 and 150 μm, more preferably between 75 and 120 μm.

7. The implantable device according to claim 1, wherein the device comprises cells in the inner space, preferably wherein the cells are cell aggregates and/or organoids, preferable wherein the cell aggregates are endocrine cells or cytokine producing cells or aggregates thereof, preferably wherein the cell aggregates are selected from islet cells, human mesenchymal stem cells, kidney cells, thyroid cells, thymic cells, testicular cells, pancreatic cells, endocrine cells, liver cells, cytokine producing cells, or preferably wherein the organoid is selected from a kidney organoid, an intestinal organoid, a pancreatic organoid, a neural organoid, a hepatic organoid, a thyroid organoid, a stomach organoid, an ovarian organoid, a prostate organoid, a splenic organoid, oesophageal organoid, a breast organoid, a bladder organoid, a lung organoid, an optic organoid, an inner ear organoid, a cardiac organoid, a biliary organoid, a salivary gland organoid, a pituitary gland organoid, a lymphoid organoid, a bladder organoid, a tongue organoid, a cerebral/brain organoid, a spinal cord organoid, a fallopian tube organoid, a lacrimal gland organoid, a skin organoid, a thymic organoid, a testicular organoid, an epithelial organoid, a gastruloid (embryonic organoid), a blastoid (blastocyst-like organoid), a retinal organoid and a hippocampal organoid.

8. The implantable device according to claim 1, wherein the device comprises two or more implantable devices stacked together and separated by spacer such as to create an open space between the individual stacked implantable devices.

9. The implantable device according to claim 1 for use as a medicament, wherein the implantable device comprises cells.

10. The implantable device according to claim 1 for use in the treatment of diabetes, wherein the implantable device comprises pancreatic islet cells or cells engineered to secrete insulin.

11. The implantable device for use according to claim 10, wherein the diabetes is type 1 diabetes.

12. The implantable device for use according to claim 10, wherein the use comprises implanting the device in a subject in need thereof, preferably wherein the implanting is at an extrahepatic site.

13. A method of constructing the implantable device as defined in claim 1, the method comprising:
providing a bottom membrane;
positioning a support structure roughly along the edges of the bottom membrane such as to leave a central space;
positioning a mesh in the central space on top of the bottom membrane;
positioning a top membrane on top of the support structure such as to cover the central space comprising the mesh as to completely enclose it creating an inner space; and
fixing the top and bottom membrane to the support structure, preferably by ultrasonic welding, heat sealing, pressure sealing, adhesives or more preferable by ultrasonic welding.

14. The method according to claim 13, wherein the top and bottom membranes are fixed to the support structure such as to leave a single opening for loading the device with cells.

15. The method for loading an implantable device with cells, the method comprising:
providing the device according to claim 1 or obtained or obtainable by the method of claim 13;
providing a cell suspension in a container;
connecting a tube such that one end is in open contact with the interior of the container with the cell suspension, and that the other end is inserted through an opening into the inner space of the implantable device; and
allowing the cells in the container to drain through the tube into the inner space of the implantable device; and optionally
closing the opening in the implantable device after removing the tube, preferably by ultrasonic welding, heat sealing, pressure sealing, adhesives or more preferable by heat sealing.
